(19) United States
(12) Patent Application Publication
(10) Pub. No.: US 2026/0218101 A1
(43) Pub. Date: Jul. 30, 2026

(54) SYSTEM, DEVICES, AND METHODS FOR FOOD SAMPLE PROCESSING FOR PATHOGEN DETECTION

(71) Applicant: PathOtrak Inc., Collage Park, MD (US)

(72) Inventors: Javier ATENCIA, Bethesda, MD (US); Ethan REGGIA, Beltsville, MD (US)

(21) Appl. No.: 19/134,457

(22) PCT Filed: Nov. 30, 2023
(86) PCT No.: PCT/US2023/081957

§ 371 Date: May 30, 2025

Related U.S. Application Data
(60) Provisional application No. 63/429,098, filed on Nov. 30, 2022.
(60) Provisional application No. 63/431,986, filed on Dec. 12, 2022.

(51) Int. Cl. C12M 1/00; C12M 1/36

(52) U.S. Cl. CPC C12M 23/14; C12M 23/38; C12M 41/48; C12M 47/02

(57) ABSTRACT

Described herein are systems, devices, and methods for pathogen detection in food or beverage samples. The methods may include introducing a food or beverage into an enclosure configured to contain one or both of a food or beverage sample, the food or beverage sample comprising the food or beverage and a growth medium, the enclosure comprising a first sealable opening at a first end of the enclosure and a second sealable opening at a second end of the enclosure, introducing the growth medium into the enclosure via a spout coupled to the second sealable opening, incubating the enclosure containing the food or beverage sample, coupling a filtration device to the spout of enclosure, applying a pressure differential across the filtration device; and extracting a retentate from the filtration device.

### CROSS-REFERENCE TO RELATED APPLICATIONS

This application claims the benefit of U.S. Provisional Patent Application No. 63/429,098, filed on Nov. 30, 2022, and U.S. Provisional Patent Application No. 63/431,986, filed on Dec. 12, 2022, the content of each of which is herein incorporated by reference in its entirety.

### STATEMENT REGARDING FEDERALLY SPONSORED RESEARCH

This invention was made with government support under NSF SBIR Phase II, award number 2127054, awarded by the National Science Foundation. The government has certain rights in the invention.

### TECHNICAL FIELD

The disclosures herein relate generally to systems, devices, and methods for food sample processing for pathogen detection.

### BACKGROUND

In the field of food processing, ensuring the safety and quality of food products is of paramount importance. The spread of foodborne illnesses may be prevented through careful monitoring of food by producers and sellers, and detection of pathogens, such as foodborne microbes, present in food processing lots. Pathogen detection processes involves enrichment, filtering of sample fluids (e.g., fluids having received a portion of a food product) to isolate and collect filtrate, and detection of e.g., foodborne microbes by polymerase chain reaction (PCR) and other known laboratory methods.

Though well established, the process of pathogen detection suffers from inefficiencies and opportunities for the introduction of contaminants. This is particularly true during enrichment and filtering. During enrichment, the food sample is mixed with bacterial nutrients in an enrichment bag and incubated for at least 22 hours to allow any bacteria that is present to increase to detectable levels. Aside from being a nearly 24-hour process, which raises questions about the utility of the process in a reality where food products are shipped instantly, current enrichment protocols also introduce contamination risks. For instance, introduction and removal of food sample and bacterial nutrients to and from the enrichment bag can result in the introduction of contaminants from the environment and/or from the user. During filtering, meaningful scale may be hindered by the inability to process food samples in parallel.

Thus, conventional methods are not optimized for scalable, rapid, consistent pathogen detection. This places consumers at risk. This may also delay sale of certain foods, which may have short shelf lives and may, therefore, be at higher risk of contamination while inefficiencies of the pathogen detection process are sorted. Accordingly, there is a need for new and improved systems, devices, and methods for food sample processing for pathogen detection.

### BRIEF SUMMARY

Described herein are systems, devices, and methods for food sample processing for pathogen detection.

According to an embodiment, the present disclosure further relates to an enrichment bag for food sample processing for pathogen detection, comprising an enclosure configured to contain one or both of a food or beverage sample, the enclosure comprising a first sealable opening at a first end of the enclosure, the first sealable opening defined by opposing side panels of the enclosure and configured to receive the food or beverage sample, and a second sealable opening at a second end of the enclosure and defined at least in part by the opposing side panels of the enclosure, and a spout coupled to the second sealable opening.

According to an embodiment, the present disclosure further relates to a method for food sample processing for pathogen detection, comprising introducing a food or beverage into an enclosure configured to contain one or both of a food or beverage sample, the food or beverage sample comprising the food or beverage and a growth medium, the enclosure comprising a first sealable opening at a first end of the enclosure, the first sealable opening defined by opposing side panels of the enclosure and configured to receive the food or beverage, and a second sealable opening at a second end of the enclosure and defined at least in part by the opposing side panels of the enclosure, introducing the growth medium into the enclosure via a spout coupled to the second sealable opening, incubating the enclosure containing the food or beverage sample and the growth medium, and extracting a fluid sample from the enclosure via the spout. In embodiments, the method further comprises detecting a presence of pathogen within the extracted fluid sample.

According to an embodiment, the present disclosure further relates to a container for enrichment bags for food sample processing for pathogen detection, comprising a container base including two or more enrichment bag receiving bays, each receiving bay being separated by a barrier, each receiving bay comprising a waste conduit outlet, and a waste conduit in fluid communication with each waste conduit outlet and with an external environment.

According to an embodiment, the present disclosure further relates to a method for food sample processing for pathogen detection, comprising introducing a food or beverage into an enclosure configured to contain one or both of a food or beverage sample, the food or beverage sample comprising the food or beverage and a growth medium, the enclosure comprising a first sealable opening at a first end of the enclosure, the first sealable opening defined by opposing side panels of the enclosure and configured to receive the food or beverage, and a second sealable opening at a second end of the enclosure and defined at least in part by the opposing side panels of the enclosure, introducing the growth medium into the enclosure via a spout coupled to the second sealable opening, incubating the enclosure containing the food or beverage sample, coupling a filtration device to the spout of enclosure, applying a pressure differential across the filtration device, and extracting a retentate from the filtration device. In embodiments, the method further comprises detecting a pathogen within the retentate extracted from the filtration device.

According to an embodiment, the present disclosure further relates to a container for enrichment bags for food sample processing for pathogen detection, comprising a container base including two or more enrichment bag receiving bays, each receiving bay being separated by a barrier, each receiving bay comprising a waste conduit outlet, and a waste conduit in fluid communication with each waste conduit outlet and with an external environment.

According to an embodiment, the present disclosure further relates to a system for food sample processing for pathogen detection, comprising an array of processing pods, each of the processing pods comprising a tubular cavity configured to receive a container, each of the processing pods comprising a fluid inlet and a fluid outlet, each of the processing pods being sealable and configured to have a pressure applied therein, and a plurality of containers, each container comprising a container base including two or more enrichment bag receiving bays, each receiving bay comprising a waste conduit outlet, and a waste conduit in fluid communication with each waste conduit outlet and with an external environment via the fluid outlet of each processing pod.

According to an embodiment, the present disclosure further relates to a method for using a system for food sample processing for pathogen detection, comprising introducing a food or beverage into an enrichment bag including an enclosure comprising a first sealable opening at a first end of the enclosure and a second sealable opening at a second end of the enclosure, wherein the first sealable opening is defined by opposing side panels of the enclosure and is configured to receive a food or beverage sample comprising the food or beverage and a growth medium, and the second sealable opening is defined at least in part by the opposing side panels of the enclosure, introducing the growth medium into the enclosure via a spout coupled to the second sealable opening, incubating the enclosure containing the food or beverage sample and the growth medium, coupling a filtration device to the spout, positioning the enrichment bag within an enrichment bag receiving bay of a container, fitting the container within a processing pod of an array of processing pods, applying a pressure differential across the filtration device, and extracting a retentate from the filtration device. In embodiments, the methods further comprises detecting a pathogen within the retentate extracted from the filtration device.

### BRIEF DESCRIPTION OF THE DRAWINGS

FIG. 1A depicts a container insertable into a processing pod of the plurality of processing pods of the enrichment bag processing tower, according to embodiments. As shown in FIG. 1A, the container comprises two main components: a base and a lid. A hinge joins the base and the lid.

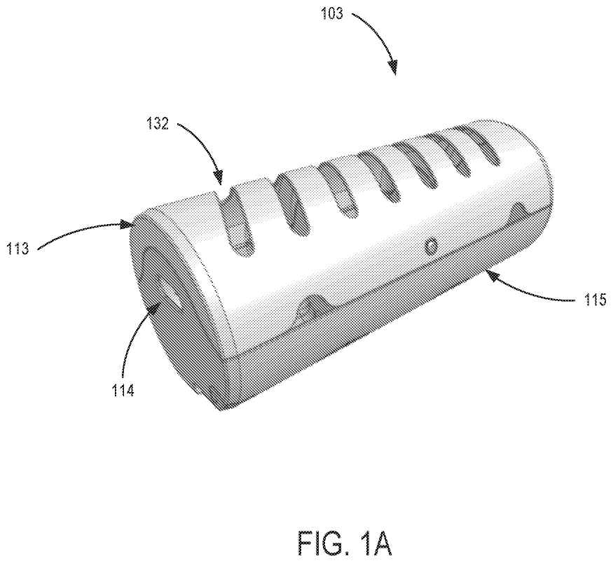

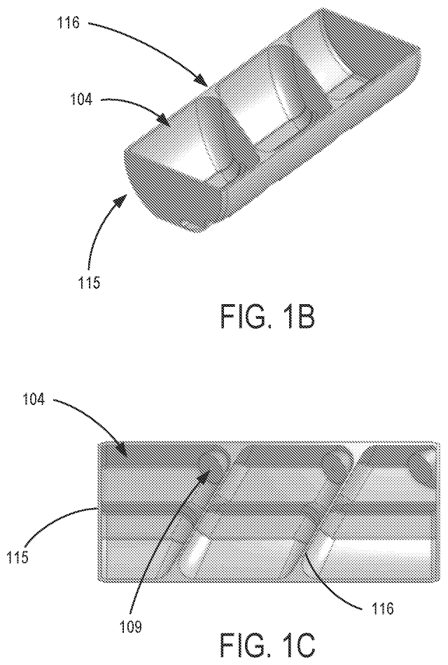

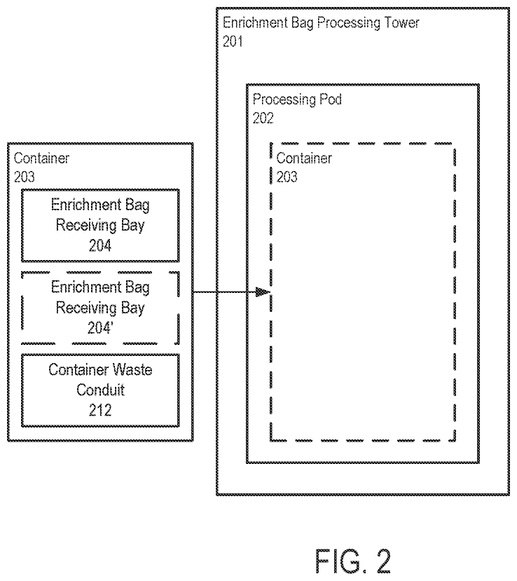

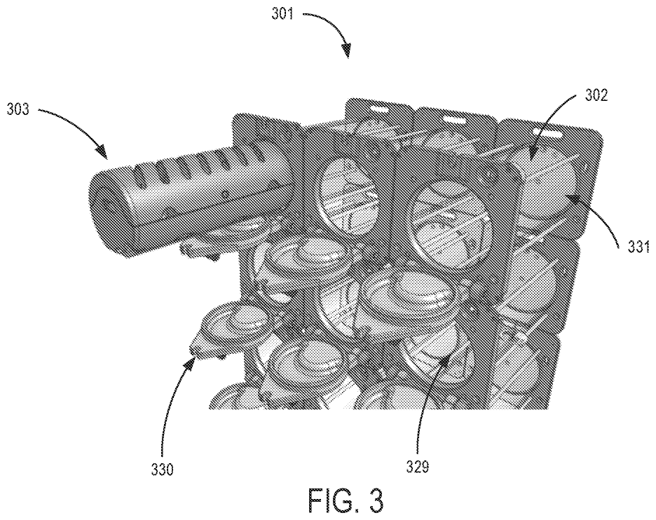

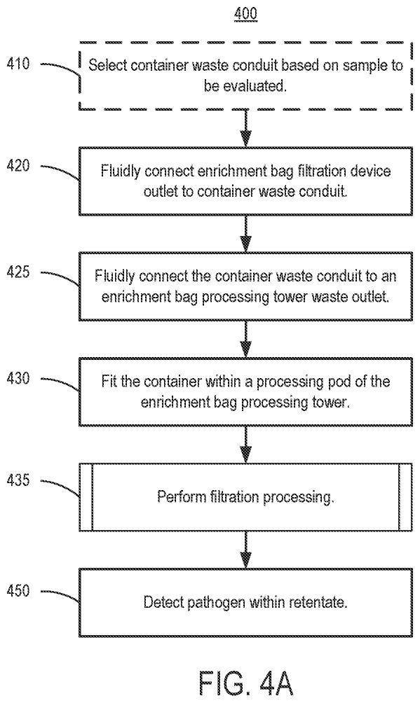

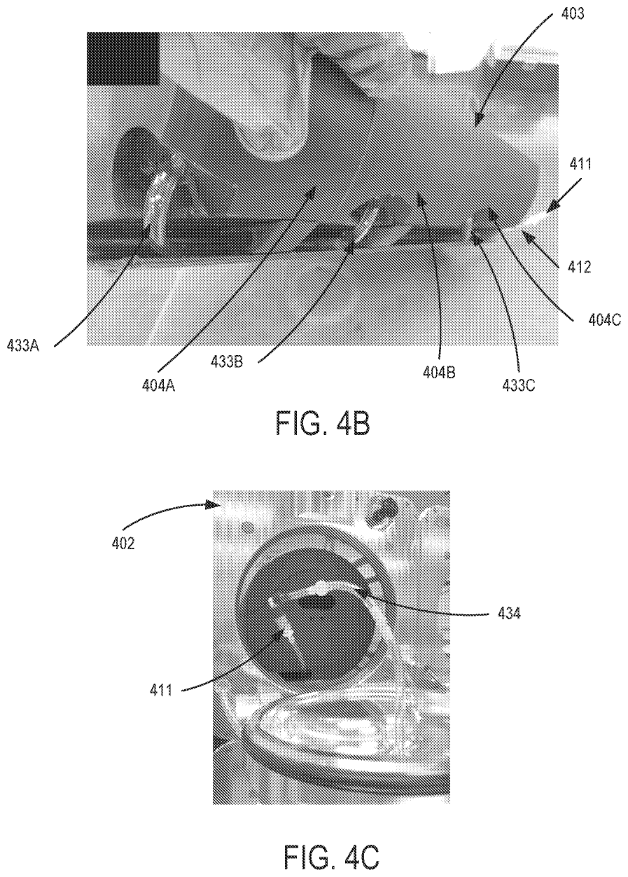

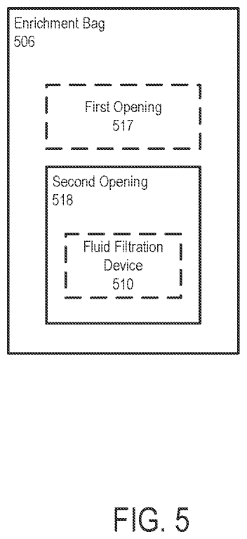

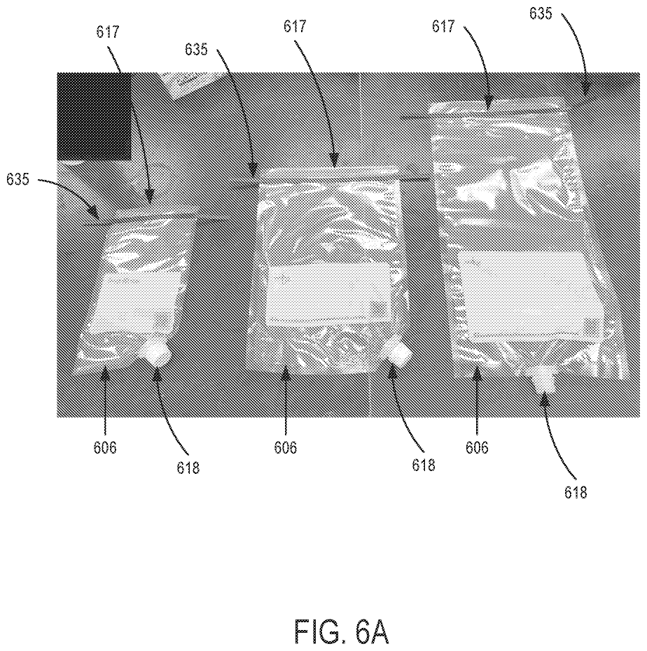

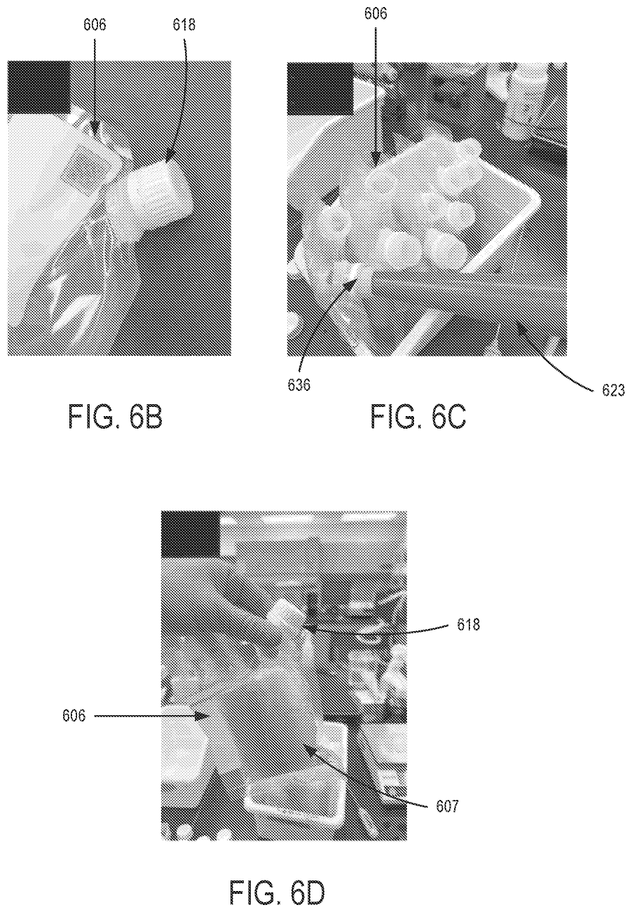

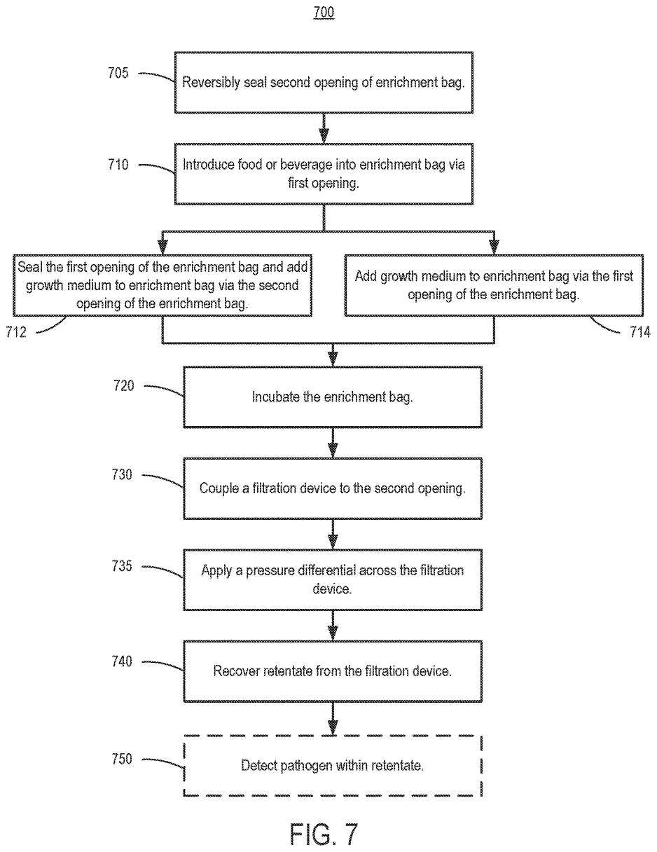

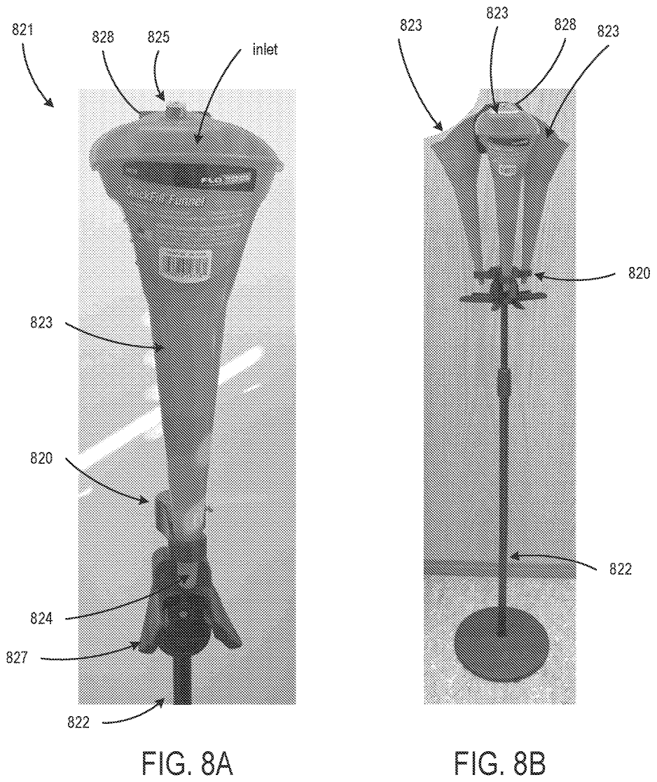

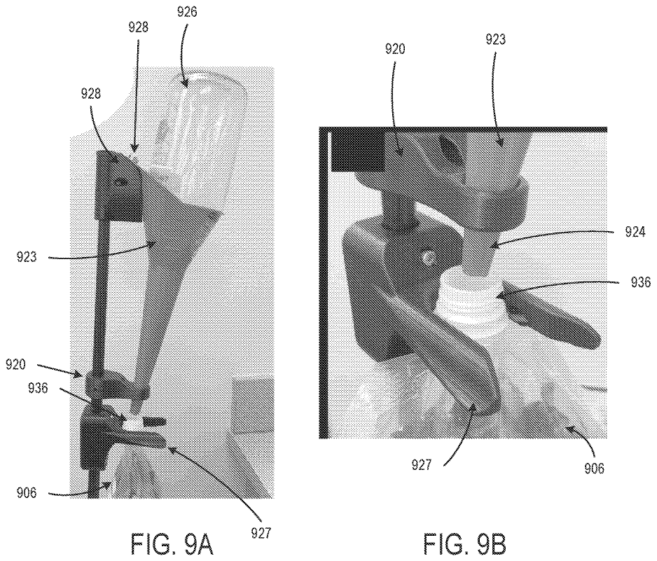

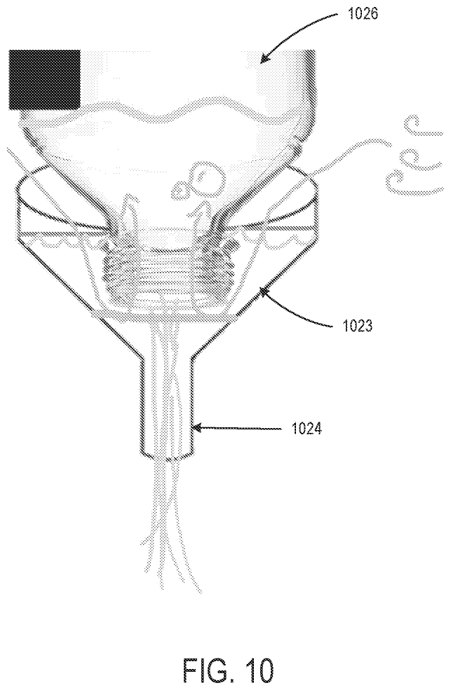

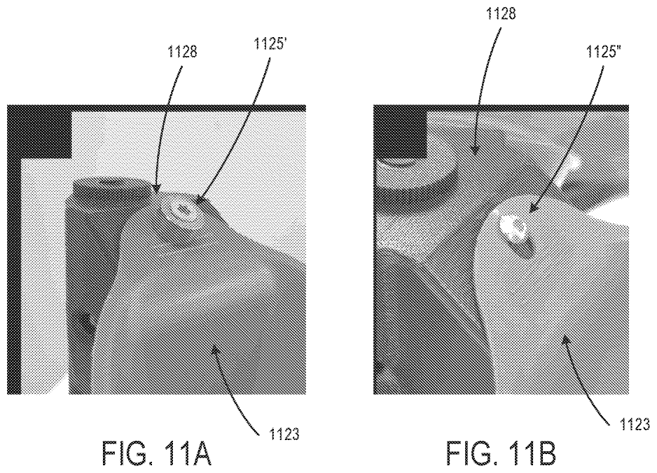

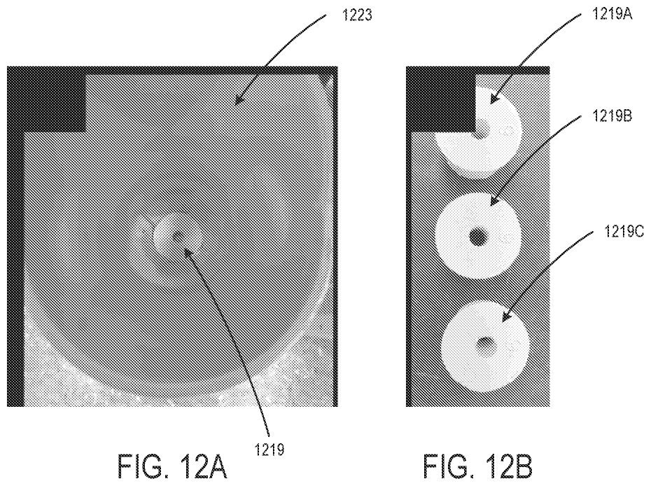

Handle holes on either end of the base facilitate carrying of the container. Apertures, or holes, in the lid permit ventilation to improve airflow. The lid can be maintained in a closed position by a snap-lock mechanism or other, similar closure mechanism. FIGS. 1B and 1C depict aspects of the container, according to embodiments. FIG. 1B depicts an exemplary arrangement of enrichment bag receiving bays. FIG. 1C provides a top view of an exemplary arrangement of enrichment receiving bays. Waste conduit outlets are shown which permit fluid connection between each enrichment bag and the container waste conduit. As appropriate, each container may comprise one, two, three, four, five, or more enrichment bag receiving bays. In an example, the container comprises three enrichment bag receiving bays, each receiving bay configured to receive an enrichment bag having a capacity of 1 liter of food or beverage sample.

FIG. 2 is a schematic of an enrichment bag processing tower, according to embodiments.

FIG. 3 depicts a perspective view of an enrichment bag processing tower, comprising a plurality of processing pods, according to embodiments. In particular, FIG. 3 conveys a drawer, or container, being inserted into one of the plurality of processing pods. Appreciating that the drawer may contain two or more enrichment bags, the enrichment bag processing tower can process a plurality of enrichment bags simultaneously.

FIG. 4A is a flow diagram of a method of using an enrichment bag processing tower for pathogen detection within a food sample, according to embodiments.

FIG. 4B depicts an aspect of a method of using an enrichment bag processing tower for pathogen detection within a food sample, according to embodiments. As shown in FIG. 4B, a combined waste conduit in a drawer is routed on the outside of the drawer housing.

FIG. 4C depicts an aspect of a method of using an enrichment bag processing tower for pathogen detection within a food sample, according to embodiments. As shown in FIG. 4C, a container waste conduit is attached to an enrichment bag processing tower waste outlet.

FIG. 5 is a schematic of an enrichment bag, according to embodiments.

FIGS. 6A-6D depict aspects of exemplary enrichment bags, according to embodiments. FIG. 6A depicts exemplary enrichment bags having different capacities. FIG. 6B depicts a second opening of an enrichment bag. FIG. 6C depicts a plurality of enrichment bags receiving a growth medium via a second opening thereof. FIG. 6D depicts an enrichment bag prepared for incubation.

FIG. 7 is a flow diagram of a method for food sample processing and pathogen detection, according to embodiments.

FIG. 8A depicts a frontal view of aspects of an enrichment bag filling station, comprising a funnel and a mounting rack, according to embodiments. The funnel hangs from a funnel locking pin at the top of the mounting rack, and the funnel outlet of the funnel is guided toward an enrichment bag bracket via a holder. FIG. 8B depicts an enrichment bag filling station comprising a plurality of enrichment bags.

FIG. 9A depicts an enrichment bag, a funnel, and a growth medium reservoir, or bottle, arranged and/or positioned on an enrichment bag filling station, according to embodiments. As shown in FIG. 9A, the weight of the enrichment bag filling station combined with the flow restriction abilities of the funnel allow the filling of the enrichment bag with growth medium to be a hands-off process. FIG. 9B depicts aspects of a funnel and an enrichment bag on the enrichment bag filling station. As shown in FIG. 9B, a funnel holder is affixed to a mounting rack of an enrichment bag filling station to direct flow from the funnel toward a second opening of the enrichment bag. Beneath the funnel holder, an enrichment bag bracket is affixed to the mounting rack of the enrichment bag filling station. Enrichment bags can be removably coupled thereto. The enrichment bag bracket comprises a depression such that the second opening of the enrichment bag sits in the depression and cannot slide or rotate during addition of growth medium.

FIG. 10 is a cartoonized diagram of flow fluid between a growth medium reservoir, or bottle, and a funnel, according to embodiments.

FIG. 11A and FIG. 11B depict funnel locking pins coupling a funnel to a mounting block. The mounting block may be positioned at a top of a mounting rack of the enrichment bag filling station. The mounting block may comprise a hole for receiving a locking pen which can be inserted through an aperture in a funnel to secure to and hang the funnel from the mounting block. As shown in FIG. 11A, the funnel locking pin may be non-removable. As shown in FIG. 11B, the funnel locking pin may be removable to enable easy cleaning of the mounting rack and the funnel.

FIG. 12A depicts an internal cavity of a funnel and a funnel fitting at a distal end thereof. FIG. 12B depicts a variety of funnel fittings having varied internal diameters such that a rate of flow therethrough can be controlled.

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “microbe” refers to any microorganisms, such as a bacteria, virus, fungi, or protozoa, cells, or other pathogens. Examples of microbes include infectious agents that may cause diseases or untoward or deleterious symptoms in an animal, such as human. As used herein, a microbe comprises bacteria, virus, fungi, and protozoa. The term “bacteria” is used herein to mean one or more viable bacteria existing or co-existing collectively in a test sample. The term may refer to a single bacterium (e.g., Aeromonas hydrophilia, Aeromonas caviae, Aeromonas sobria, Streptococcus uberis, Enterococcus faecium, Enterococcus faecalis, Bacillus sphaericus, Pseudomonas fluorescens, Pseudomonas putida, Serratia liquefaciens, Lactococcus lactis, Xanthomonas maltophilia, Staphylococcus simulans, Staphylococcus hominis, Streptococcus constellatus, Streptococcus anginosus, Escherichia coli, Staphylococcus aureus, Mycobacterium fortuitum, and Klebsiella pneumonia), a genus of bacteria (e.g., streptococci, pseudomonas and enterococci), a number of related species of bacteria (e.g., coliforms), an even larger group of bacteria having a common characteristic (e.g., all gram-negative bacteria), a group of bacteria commonly found in a food product, an animal or human subject, or an environmental source, or a combination of two or more bacteria listed above.

In other embodiments, the term “microbe” also encompasses pathogens. The term “pathogen” as used herein refers to any microorganism that can cause disease. One exemplary embodiment of pathogen is foodborne pathogen that are present in the food and are the cause of major diseases, such as food poisoning. Exemplary of common foodborne pathogens include Salmonella, E. coli O157:H7, E. coli STEC, Listeria, Campylobacter, Clostridium botulinum, Staphylococcus aureus, Shigella, Toxoplasma gondii, Vibrio vulnificus, Norovirus, and Legionella.

In embodiments, the present disclosure relates to a container configured to house one or more enrichment bags. In variations, the container houses two or more enrichment bags and enables parallel processing. Processing includes filtering a perfusate (e.g., growth medium or broth) from each enrichment bag to isolate pathogens within a filtration device coupled to each enrichment bag. Fluid flow from the enrichment bag and through the filtration device can be controlled by a pressure applied thereto and by fluid conduits that receive a filtrate. For instance, waste lines or fluid conduits outlet from each of the enrichment bags can be fluidically connected within the container and linked to a primary outlet. A dimension of the fluid conduits can provide a natural mechanism for regulating flow through each filtration device.

Parallel processing means that multiple enrichment bags, once loaded into a container, can be processed together as the container is moved to and from an incubator and to and from a processing chamber. Each processing chamber, or pressure pod, may universally apply a pressure regime to the enrichment bags. Each enrichment bag may be coupled to a respective filtration device.

In embodiments, the present disclosure relates to an enrichment bag for conducting incubation and handling of specimens for food and beverage testing. The enrichment bag may comprise at least one opening for introducing growth medium. The at least one opening may be configured for introduction or removal of specimen samples from the enrichment bag. The at least one opening may two openings configured for introduction or removal of specimen samples from the enrichment bag. In embodiments, the enrichment bag may be coupled to a fluid filtration device and isolation of pathogens from a food or beverage sample can be performed.

In embodiments, the present disclosure relates to an enrichment bag filling station comprising a funnel having a funnel outlet and a mounting rack configured to hold an enrichment bag and the funnel. In an example, a bottle of growth medium comprising bacterial nutrients can be positioned within (i.e., inverted into) the funnel and the growth medium can flow in a controlled manner from the bottle to the enrichment bag via the funnel outlet. The funnel outlet may have a variable diameter. In other words, to control the rate of flow of growth medium between the bottle and the enrichment bag, the diameter of the funnel outlet can be modified. The mounting rack is configured to position a mouth or opening of the enrichment bag out of contact with the funnel outlet but in proximity such that growth medium can be transferred without splashing or spilling. The enrichment bag filling station may be left unattended during filling.

### Enrichment Bag Processing Tower and Container

Until now, the enrichment step of pathogen detection testing has been carried out by incubating samples of food immersed in a flask of bacterial growth media for upwards of 24 hours. Using the methods described here, incubation time of a single food or beverage sample can be reduced from 24 hours to as low as five hours, yielding significant time savings compared to traditional incubation methods. However, to exploit this enhanced speed and further expedite processing of food or beverage samples for accurate pathogen detection, it is necessary to reduce cross-contamination and to increase the scale at which food or beverage samples can be processed.

Cross-contamination is a major concern in a food safety testing setting because false positive results can be highly costly if product is needlessly disposed of. Enrichment bags are often flexible for handling and processing but can be easily pierced. Keeping samples separate is essential for traceable and valid test results. The less contact the bags have with one another, the better. Thus, the present disclosure provides a container that maintains division of food or beverage samples during incubation and during processing to remove the need of the lab technicians to devise their own systems of isolation.

Currently available systems are not readily applicable within high throughput approaches. For instance, when ready for processing, a filtration device can be affixed to an enrichment bag's spout. “Enriched” growth medium, or growth medium that has been in contact with a food or beverage within the enrichment bag and may have pathogen therein, must pass through the filtration device to capture possible pathogens. During filtration, a filtrate flows through a waste outlet of each filtration device, into a container waste outlet, and to a waste container outside the processing pod for disposal. Current systems fluidly connect a single enrichment bag to a single waste line within a processing pod. Thus, a single enrichment bag can be processed within each processing pod per round of processing. Because the processing pods are difficult to construct and must be airtight, however, it is not practicable to construct dozens of processing pods for a single lab. Neither is it favorable to make numerous perforations in a chassis to allow for multiple waste lines. Thus, the present disclosure provides a method for processing multiple enrichment bags within a single processing pod simultaneously, thereby enabling high throughput testing. Waste fluid from multiple enrichment bags can be routed to the exterior of the processing pod during pressurization of the multiple bags so that the waste of many enrichment bags can be pooled and exited through a single waste conduit.

Accordingly, the cartridge and system of the present disclosure streamlines processing, facilitates laboratory cleanliness and sample tracking by grouping and individualizing samples, and increases the density of tests that may be run simultaneously. Moreover, an operator's time spent planning, organizing, and cleaning is reduced, thereby reducing labor costs associated with performing food safety tests.

Turning now to FIGS. 1A through 1C, aspects of a container 103 for processing a food or beverage sample for pathogen detection are shown. In embodiments, the container 103 may include a base 115 comprising one or more enrichment bag receiving bays 104. Each of the one or more enrichment bag receiving bays 104 may be configured to receive an enrichment bag. Each receiving bay 104 may be isolated within the container 103 by at least one barrier 116. The at least one barrier 116, along with sidewalls of the base 115, may physically isolate each receiving bay 104 and prevent cross-contamination between food or beverage samples in adjacent receiving bays 104. Each receiving bay 104 may comprise a receiving bay waste outlet 109. The receiving bay waste outlet 109 may be fluidly coupled to a container waste outlet (not shown) by a container waste conduit (not shown).

In embodiments, the container 103 may comprise a lid 113 to enclose the container 103 and prevent enrichment bags within receiving bays 103 of the base 115 from moving when the container 103 is carried horizontally or vertically. In embodiments, apertures or openings 132 in the lid 113 enable air circulation in and out of the container 103.

In embodiments, the base 115 of the container 103 may comprise at least one handle 114 to facilitate carrying the container 103.

In embodiments, the container 103 may comprise at least one container waste conduit to couple each receiving bay waste outlet 109 to at least one container waste outlet. In embodiments, the container 103 comprises a container waste conduit for each receiving bay waste outlet 109. In embodiments, the container 103 comprises at least one connector that fluidly couples each receiving bay waste outlet 109 to a single container waste conduit which is fluidly coupled to the container waste outlet. The container waste conduit may be disposed within the container 103 at any location such that each receiving bay waste outlet 109 is fluidly coupled to the container waste outlet. In an example, the container waste conduit may be arranged at a bottom of the base 115. To avoid entanglement with enrichment bags, the container waste conduit may be contained within a housing disposed on an internal surface of the base 115. Any location of the container waste conduit housing is suitable so long as the fluid connection is provided and handling of the container 103 is preserved. In embodiments, each receiving bay waste outlet 109 may comprise a one-way valve to prevent return flow from the container waste conduit, or a connector thereto, and each enrichment bag.

The container waste conduit acts to dampen flow through a filtration unit (connected between each enrichment bag and a receiving bay waste outlet 109. When filtration begins and flow through the filtration device would otherwise be high, the container waste conduit, which may be sized accordingly, restricts the amount of fluid that can pass through the filtration device. Later, after substantial filtration has already occurred, the size of the container waste conduit is no longer a constraint and filtrate can pass through the container waste conduit as waste without impediment. In embodiments, the container waste conduit can be sized according to a number, size, and desired flow rate of the enrichment bags to be processed. Sizing of the container waste conduit can be informed by, among other things, application of Bernoulli's principle.

In embodiments, the container waste conduit can have a length of at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 15 mm, at least about 20 mm, at least about 30 mm, at least about 40 mm, at least about 50 mm, at least about 100 mm, at least about 250 mm, at least about 500 mm, or at least about 100 mm.

In embodiments, the container waste conduit can have a diameter of at least about 0.5 mm, at least about 1 m, at least about 1.5 mm, at least about 2 mm, at least about 2.5 mm, at least about 3 mm, at least about 3.5 mm, at least about 4 mm, at least about 4.5 mm, or at least about 5 mm.

In embodiments, the container waste conduit may comprise at least one material selected from the group consisting of polyvinylchloride, acrylonitrile butadiene styrene, polyvinylidene difluoride, polyethylene terephthalate, polypropylene, polytetrafluoroethylene, expanded polytetrafluoroethylene, low-density polyethylene, and high-density polyethylene.

Generally, each receiving bay 104 may be any size or shape sufficient to hold an enrichment bag of a known size or capacity. For instance, depending on the known size or capacity of the enrichment bag, the number of barriers 116 may be modified such that the base 115 comprises zero, one, two, three, four, five, six, seven, eight, nine, or ten receiving bays 104. In an example, when each enrichment bag has a known capacity of 1.5 kg, the base 115 may comprise zero barriers 116 and may only be able to accommodate a single enrichment bag. In another example, when each enrichment bag has a known capacity of 375 g, the base 115 may comprise two barriers 116 to separate the base 115 into three enrichment bag receiving bays 104, each configured to house a single enrichment bag. In still another example, when each enrichment bag has a known capacity of 25 g or 250 ml, the base 115 may comprise nine barriers 116 to separate the base 115 into 10 enrichment bag receiving bays 104, each configured to house a single enrichment bag. As can be appreciated in these examples, when a plurality of enrichment bags of a same size is processed, the barriers 116 may be evenly spaced within the base 115 to accommodate the plurality of enrichment bags. In other examples, the plurality of enrichment bags to be processed are of different sizes, and the barriers 116 within the base 115 may be spaced, accordingly.

FIG. 2 is a schematic of a system for processing food or beverage samples, comprising an enrichment bag processing tower 201 and at least one container 203. The enrichment bag processing tower 201 may comprise at least one processing pod 202. As shown in FIG. 3, in embodiments, the at least one processing pod 202 comprises a plurality of processing pods 202. Each of the at least one processing pod 202 may be configured to receive a container 203. Direction lines in FIG. 2 indicate that the container 203 may be positioned within or fitted within the at least one processing pod 202. Dashed lines indicate the transience of the container 203 within the at least one processing pod 202. The container 203, which may be similar to the container 103 described with reference to FIG. 1A through FIG. 1C, may comprise at least one enrichment bag receiving bay 204 and a container waste conduit 212. Dashed lines around enrichment bag receiving bay 204′ indicate that the container 203 may optionally comprise a plurality of receiving bays, shown in FIG. 1B and FIG. 1C.

In addition to the above-described containers comprising multiple enrichment bags, the present disclosure provides for further parallelization. As shown in FIG. 3, an enrichment bag processing tower 301 may comprise a plurality of processing pods 302. The plurality of processing pods 302 may be arranged in a manner suitable to handling by an operator. For instance, the plurality of processing pods 302 may be arranged in a geometric array. Each of the at least one processing pod 302 may comprise a cavity 329. The cavity 329 may be any size or shape for receiving a container 303. For instance, the cavity 329 may be rectangular, circular, tubular, or other shape. The cavity 329 may have comprise any dimensions appropriate for receiving a container 303. In embodiments, the cavity 329 may be tubular to accommodate a tubular shape of the container 303 and to facilitate convenient handling of the container 303.

In embodiments, fitting the container 303 within the at least one processing pod 302 may comprise fluidly connecting the container 303 to the at least one processing pod 302 and sealing the at least one processing pod 302 by securing a latch or door 330. As will be described in more detail with reference to FIG. 4A through FIG. 4C, fitting the container 303 within the at least one processing pod 302 comprises inserting the container 303 into the processing pod 302, fluidly connecting a container waste conduit of the container 303 to fittings on a rear wall 331 of the processing pod 302, and sealing the processing pod 302 by securely closing the door 330.

In embodiments, after securing the container 303 within the processing pod 302, one or more of a plurality of actions may occur. For instance, the processing pod 302 may be configured for incubation of the food or beverage sample and, as such, incubation may be performed. To this end, the rear wall 331 of the processing pod 302 may comprise a heating element and temperature sensor, a humidifying element and humidity sensor, a gaseous sensor and mixed gas lines, and the like. Additionally, or alternatively, the processing pod 302 may be configured for filtration of the food or beverage sample. To this end, as will be described below, the rear wall 331 of the processing pod 302 may comprise fittings to connect the cavity 329 to a pressure regulator. Moreover, the rear wall 331 of the processing pod 302 may comprise a processing pod waste outlet in fluid communication with a container waste outlet to allow for filtration of the food or beverage sample. The container waste outlet may be fluidly connected to the processing pod waste outlet, which may be connected to an enrichment bag processing tower waste outlet, by a luer-lock, barbed connections, snap-in mechanisms, and the like.

With reference now to FIG. 4A through FIG. 4C, a method of using an enrichment bag processing tower for pathogen detection will be described.

Initially, but optionally, at step 410 of method 400, a container waste conduit can be selected according to a type and amount of food or beverage to be evaluated. For instance, as discussed above, a size, shape, and material of the container waste conduit may be determined by the type and amount of food or beverage to be filtered. In embodiments, the type of food or beverage may be leaf vegetables, chicken, beef, pork, fish, root vegetables, cheese, dairy, swab, cloth, spices, fruit, broth, milk, water, and the like, or any other food or beverage requiring evaluation for presence of pathogens. When the food or beverage is a swab or cloth, the swab or cloth may have been in contact with e.g., beef, pork, and veggies or may have been used as an environmental swab to obtain environmental samples. Understanding the type of food or beverage to be evaluated, and their impact on expected fluid flow rate, aids in the determination of the required characteristics of the container waste conduit.

In embodiments, a size, shape, and material of the container waste conduit may be based solely on the container to be used. For instance, when a container comprises three equally sized enrichment bag receiving bays, a corresponding size, shape, and material of container waste conduit can be used. When the container features are known, the container waste conduit may be integrally formed with the container based on the size of the receiving bays therein prior to user interaction. In another instance, when a container comprises 10 equally sized enrichment bag receiving bays, the container waste conduit may be a shunt, effectively providing no resistance to flow between each receiving bay waste outlet and the container waste outlet.

At step 420 of method 400, a waste outlet of each enrichment bag can be fluidly connected to a container waste conduit via a respective receiving bay waste outlet. The waste outlet of each enrichment bag may be, for instance, an outlet of a filtration device connected to a spout of each enrichment bag. FIG. 4B depicts a container 403 comprising receiving bay waste outlet connectors 433A, 433B, 433C connecting a respective receiving bay waste outlet (of receiving bay 404A, 404B, 404C) with a container waste conduit 412.

At step 425, the container waste conduit outlet can be fluidly connected to the enrichment bag processing tower waste outlet via a processing pod waste outlet. As shown in FIG. 4B and FIG. 4C, a container waste conduit outlet 411 can be fluidic connected with a processing pod waste outlet 434 of the processing pod 402.

With the container fluidly connected to the processing pod and, thus, to the enrichment bag processing tower, the container may be fitted within the processing pod at step 430 of method 400. Fitting the container within the processing pod may comprise sliding the container into a cavity of the processing pod and sealing the cavity by latching a door thereof.

Filtration may then be performed on the food or beverage sample at sub process 435 of method 400. Generally, filtration includes flowing perfusate through the filtration device connected to each enrichment bag, disposing of filtrate as waste, and recovering retentate from the filtration device as a potentially pathogen containing sample. As described herein, filtration includes the application of a pressure differential across the filtration device. In embodiments, a pressure differential across the filtration device, or between each enrichment bag and container waste conduit outlet, may be equal to or greater than about 4 pounds per square inch (psi). In embodiments, the pressure differential may be between about 4 psi and about 10 psi, between about 10 psi and about 15 psi, between about 15 psi and about 20 psi, or between about 20 psi and about 30 psi. In embodiments, the pressure differential may be equal to or greater than about 20 psi, equal to or greater than about 30 psi, and/or equal to or greater than about 40 psi. In embodiments, the pressure differential is between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4.5 psi and about 5.5 psi. Similar methods of filtration, such as those described in U.S. patent application Ser. No. 17/603,248, which is incorporated herein in its entirety by this reference, may be deployed herein as appropriate.

As introduced above, after filtration is performed, the container may be removed from the processing pod and each filtration device connected to each enrichment bag may be retrieved. The retentate, which is the portion of the perfusate which did not pass through the filtration device, may then be collected, or recovered, from the retrieved filtration devices. To collect retentate, the filtration device, or filter(s) thereof, can be introduced into a tube or container with a suitable liquid and sonicated or otherwise have shear applied to it. Subsequently, the retentate is re-suspended in the liquid. Counterflow elution, or backwashing, of a liquid through the filtration device, or filter(s) thereof, may alternatively be deployed. In an embodiment, an appropriate volume of liquid, such as water or a buffered solution, can be pumped backwards, in the direction opposite to the filtration direction used previously, to release the retentate from the filtration device.

In embodiments, about 1-10 ml of deionized water is pumped backwards. In other embodiments, about 1-5 ml of deionized water is pumped backwards. The volume of the liquid can be adjusted based on various factors, for example, the size and/or pore size of the filter(s). In an exemplary embodiment, about 1.5 ml of deionized water is pumped backwards to collect the retentate from the filter(s), which may be about 25 mm in diameter. In other embodiments, the liquid can be, but is not limited to culture medium, a buffered solution, and/or mixtures thereof. Pathogens, which may be microbes, within the retentate can be collected from the liquid.

In embodiments, recovering pathogens from the collected retentate encompasses introducing the collected retentate into a vial or tube suitable for centrifugation. In embodiments, the speed of centrifugation is between about 10 G to about 100 G. In an embodiment, the speed of centrifugation is about 50 G or is about 22 G. In an embodiment, the speed of centrifugation is greater than about 100 G or is less than about 10 G. After centrifugation, the supernatant may be discarded and a pellet from the centrifugation can be processed for detection of pathogens at step 450 of method 400.

Detection at step 450 of method 400 comprises detecting by molecular methods such as selectively culturing the microbe or by polymerase chain reaction (PCR). In embodiments, the detecting can be done by a real-time PCR (RT-PCR), by a quantitative real-time PCR (qRT-PCR), or by a quantitative PCR (qPCR). In embodiments, the processed retentate comprising the pathogen may be assayed for contamination by any other PCR-based detection techniques not listed here. In another embodiment, the processed retentate comprising the pathogen may be assayed by plating on selective media for specific pathogens.

Detection during method 400 may provide information to a producer of the food or beverage to quarantine or otherwise remove a product containing the food or beverage from the marketplace. Importantly, the present method permits such a determination to be made in a fraction of the time previously required, enabling producers to readily remove harmful products from the marketplace.

### Enrichment Bag

Enrichment bags for food safety collection are used to incubate a food or beverage with growth medium to encourage proliferation of microorganisms or pathogens to detectable levels. To this end, current methods for processing a food or beverage within an enrichment bag for pathogen detection are tedious and prone to contamination. Current methods require a weight of food or beverage (typically 1 g, 10 g, 25 g, 60 g, 325 g, 375 g, or 1500 g) to be loaded into enrichment bags, which are then impermanently closed and shipped to the laboratory for analysis. These enrichment bags usually have an affixed wire tie near a wide opening, allowing closure by folding the opening on itself several times and tying the tie. At a lab, a technician opens each enrichment bag and introduces growth medium (typically 1:3 or 1:10 weight/volume) and then recloses the enrichment bag for incubation. As noted, incubation grows pathogenic bacteria, such as listeria, salmonella, and the like, to detectable levels, which can be over 1,000,000 pathogens per milliliter of enrichment media. After the pathogenic population grows sufficiently, the technician opens the bag again to extract an aliquot (typically 1 mL) with a micropipette for further analysis. This process is tedious to perform, dangerous to the technician/operator, and hazards cross-contamination between the micropipette and other samples. Additionally, ineffective closing and handling of the bag may cause pathogenic liquid to splash out and taint nearby surfaces during unfolding.

Accordingly, the present disclosure provides an enrichment bag that simplifies processing and reduces the risks for contamination. For instance, the enrichment bags of the present disclosure comprise two openings. First, a food or beverage is loaded into the enrichment bag via a first opening of the enrichment bag, as would normally be done. Once the enrichment bag arrives at the lab, however, a technician only needs to open a second opening (e.g., threaded cap of a spout) to introduce growth medium. As such, handling and processing is significantly easier and faster, eliminating the need to unfold the enrichment bag with the food or beverage and fold it again after the growth medium is added. Moreover, the enrichment bag simplifies incubation. Traditionally, a lab technician needs to perform analysis both before incubation and after incubation to test for the presence of pathogens, thus requiring more folding and unfolding of the enrichment bag and more opportunities for contamination. According to the devices and methods herein, testing before, during, or after incubation becomes simpler and safer. For instance, when the second opening comprises a spout, accessing the food or beverage sample requires only opening a small and rigid threaded spout instead of a large, several-times-folded plastic bag.

As described above, enrichment bags of the present disclosure can be positioned within receiving bays of a container and processed within processing pods of an enrichment bag processing tower. FIG. 5 depicts a schematic of an enrichment bag 506 of the present disclosure. Each enrichment bag 506 of the present disclosure may comprise a first opening 517 at a first end of the enrichment bag 506, a second opening 518 and a second end of the enrichment bag 506, and a void therebetween defined by opposing side walls of the enrichment bag 506. The first opening 517 may be optional, however, remaining discussion herein will consider the first opening 517 to be included. The second opening 518 may be optionally coupled to a filtration device 510. The filtration device 510 may be positioned within the enrichment bag 506 without being coupled to the second opening 518. However, as was described above and as will be described below, the filtration device 510 is considered to be fluidly coupled to the second opening 518. The filtration device 510 may be fluidly coupled to the second opening 518 during manufacturing or may be coupled to the second opening 518 by a user during performance of a method, such as method 400 of FIG. 4A.

In embodiments, the enrichment bag 506 of FIG. 5 is a sealable or re-sealable bag. The first opening 517 may be temporarily or permanently sealable with a tin tie, a zipper mechanism, a hook and loop mechanism, heat, and the like. In embodiments, the first opening 517 comprises at least a portion of a dimension of the first end of the enrichment bag 506. In other words, the first opening 516 may extend along the first end of the enrichment bag 506 from a left edge to a right edge of the enrichment bag 506 or may extend only a portion therebetween. The portion may be any portion along the length of the first end between the left edge and the right edge. For instance, the portion comprising the first opening 517 may be evenly spaced between the left edge and the right edge or may favor one or the other of the left edge and the right edge. The portion of the first end comprising the first opening 517 may have an inner dimension. The inner dimension may be an inner width or an inner diameter. For instance, if a length between the left edge of the enrichment bag 506 and the right edge of the enrichment bag 506 at the first end is 10 cm, the first opening 517 may occupy a 5 cm portion thereof. In embodiments, the first opening 517 may have an inner width having a distance of between about 1 cm and about 10 cm, between about 2 cm and about 9 cm, between about 3 cm and about 8 cm, between about 4 cm and about 7 cm, or between about 5 cm and about 6 cm. Additionally, or alternatively, the first opening 517 may have an inner width having a distance of between about 5% and about 95% of the length between the left edge of the enrichment bag 506 and the right edge of the enrichment bag 506 at the first end of the enrichment bag 506, which be may be referred to herein as an outer width of the first opening 517. In embodiments, the first opening 517 may have a distance of between about 5% and about 95%, between about 10% and about 90%, between about 15% and about 85%, between about 20% and about 80%, between about 25% and about 75%, between about 30% and about 70%, between about 35% and about 65%, between about 40% and about 60%, and/or between about 45% and about 55% of the length between the left edge and the right edge at the first end of the enrichment bag 506.

In embodiments, the second opening 518 may be temporarily or permanently sealable with a tin tie, a zipper mechanism, a hook and loop mechanism, heat, and the like. In embodiments, the second opening 518 comprises at least a portion of a dimension of the second end of the enrichment bag 506. In other words, the second opening 518 may extend along the second end of the enrichment bag 506 from the left edge to the right edge of the enrichment bag 506 or may extend only a portion therebetween. The portion may be any portion along the length of the second end between the left edge and the right edge. For instance, the portion comprising the second opening 518 may be evenly spaced between the left edge and the right edge or may favor one or the other of the left edge and the right edge. In embodiments, the second opening 518 may be on a corner of the second end of the enclosure. The portion of the second end comprising the second opening 518 may have an inner dimension. The inner dimension may be an inner width or an inner diameter. For instance, if a length between the left edge of the enrichment bag 506 and the right edge of the enrichment bag 506 at the second end is 10 cm, the second opening 518 may occupy a 5 cm portion thereof. In embodiments, the second opening 518 may have an inner width having a distance of between about 1 cm and about 10 cm, between about 2 cm and about 9 cm, between about 3 cm and about 8 cm, between about 4 cm and about 7 cm, or between about 5 cm and about 6 cm. The second opening 518 may have an inner diameter of between about 1 mm and about 1 cm, between about 5 mm and about 75 mm, between about 10 mm and about 70 mm, between about 15 mm and about 65 mm, between about 20 mm and about 60 mm, between about 25 mm and about 55 mm, between about 30 mm and about 50 mm, and/or between about 35 mm and about 45 mm. Additionally, or alternatively, the second opening 518 may have an inner width having a distance of between about 5% and about 95% of the length between the left edge of the enrichment bag 506 and the right edge of the enrichment bag 506 at the second end of the enrichment bag 506, which may be referred to herein as an outer width of the second opening 518. In embodiments, the second opening 518 may have a distance of between about 5% and about 95%, between about 10% and about 90%, between about 15% and about 85%, between about 20% and about 80%, between about 25% and about 75%, between about 30% and about 70%, between about 35% and about 65%, between about 40% and about 60%, and/or between about 45% and about 55% of the length between the left edge and the right edge at the second end of the enrichment bag 506. In embodiments, the second opening 518 comprises an inner dimension that is less than an inner dimension of the first opening 517.

In embodiments, the second opening 518 comprises a spout and inner surfaces of the second opening 518 are sealable about a perimeter of the spout. To this end, an inner dimension of the second opening 518 may be about equal to an outer dimension of the spout, wherein the outer dimension may comprise an outer width or outer diameter of the spout. The spout may comprise a spout neck, which may be configured to permit the enrichment bag 506 to releasably hang from the spout neck (as will be described later with reference to FIGS. 8A-9B). The spout may comprise a tethered or removable cap that is coupled to the spout when food or beverage is being added to the enrichment bag 506, when incubation is occurring, and the like. In this way, the second opening 518 is configured to be temporarily sealed via the removable cap. As required by a particular application, the spout may comprise a shape selected from the group consisting of circular, rectangular, and elliptical. In embodiments, the spout and/or the tethered removable cap comprise a material selected from the group consisting of polypropylene, polyethylene, high-density polyethylene, and low-density polyethylene. A diameter and a shape of each spout can be altered to fit different applications. In embodiments, the spout and the tethered or removable cap are configured to be releasably coupled by a rotational coupling or a press fit coupling. For instance, the spout can be threaded or unthreaded, and if threaded, can be made as a male or a female connection. Different varieties of connection types could be designed onto the spout as well, including luer-slips or barbed/flared tube connectors.

In embodiments, when the spout comprises a first spout and the removable cap comprises a first removable cap, the enrichment bag further comprises a third opening comprising an inner dimension that is less than an inner dimension of the first opening 517 and a second spout coupled to the third opening and comprising a second removable cap. The third opening may be sealable and may be configured to be sealed about a perimeter of the second spout.

In embodiments, the enrichment bag 506 comprises one or more materials selected from the group consisting of polypropylene, polyethylene, and low-density polyethylene. In embodiments, the enrichment bag 506 is configured to receive a food or beverage sample comprising growth medium and up to about 25 grams, up to about 350 grams, and/or up to about 1500 grams of leaf vegetables, chicken, beef, pork, fish, root vegetables, swab, such as an environmental swab, cloth, cheese, dairy, spices, fruit, broth, milk, water, and the like.

In embodiments, wherein the second opening 518 is a spout, the spout permits the design of interfacing components that permit the enrichment bag 506 to be compatible with other devices, including automated filling systems, movement throughout factory processing lines, and the like. An exemplary filling system that exploits the spout design is described below with reference to FIGS. 8A-9B.

In embodiments, and as noted above, a position of the first opening 517 and the second opening 518 can be altered to be located at an edge, at a corner, or on one of the faces of the enrichment bag 506. Additional spouts can be added to facilitate the simultaneous addition of numerous liquids.

FIG. 6A depicts three exemplary enrichment bags 606. Each of the enrichment bags, though of varying sizes and capacities, feature two openings (a first opening 617 having a closure 635, depicted as a tin tie, and a second opening 618 depicted as comprising a spout). From left to right, the first enrichment bag is configured to hold about 25-gram samples, about 350-gram samples, and about 1500 grams, respectively. In embodiments, the actual capacity is a function of the density of the sample specimens introduced. As shown, each exemplary enrichment bag 606 may be manufactured such that a spout, or the at second opening 618 on a second end of the enrichment bag 606, can be at any location on the second end. FIG. 6B provides a magnified image of a second opening 618 of an exemplary enrichment bag 606. The second opening 618 may be a spout and a removable cap may be threadably engaged on the spout. As shown, a base of the spout is sealed into the second opening 618 of the enrichment bag 606 at the second end thereof to maintain sterility. As shown in FIG. 6C, multiple enrichment bags 606, comprising food or beverage, may be filled with growth medium 607 via a funnel 623. In variations, a funnel 623 may be introduced into a spout 636 of one of the enrichment bags 606 to facilitate addition of the growth medium 607 to a food or beverage. In other variations, growth medium is added to the enrichment bag 606 without contact with the enrichment bag 606, as will be described later. Such non-contact addition can be achieved by e.g., use of a funnel positioned proximate but not in contact with the spout of the enrichment bag 606.

FIG. 7 is a flow diagram of a method for food sample processing and pathogen detection, according to embodiments. Method 700 of FIG. 7 will be described below in view of FIGS. 5-6D.

At step 705 of method 700, a second opening of an enrichment bag may be reversibly sealed. For example, the second opening of the enrichment bag may be a spout and the reversible seal may be coupling of a removable cap.

At step 710 of method 700, food or beverage can be introduced into the enrichment bag via the first opening. As described above, the food or beverage sample may comprise up to about 25 grams, up to about 350 grams, and/or up to about 1500 grams of leaf vegetables, chicken, beef, pork, fish, root vegetables, swab, such as an environmental swab, cloth, cheese, dairy, spices, fruit, broth, milk, water, and the like.

Next, method 700 progresses to either step 712 or step 714. When growth medium is added to the enrichment bag via the second opening, method 700 progresses to step 712. At step 712, the first opening of the enrichment bag is sealed, the second opening of the enrichment bag is unsealed, and growth medium is added to the enrichment bag via the second opening. When growth medium is added to the enrichment bag via the first opening, method 700 progresses to step 712, and growth medium is directly added to the enrichment bag via the first opening. The first opening is then sealed.

After either step 712 or step 714 of method 700, the enrichment bag may be incubated at step 720. The incubating may be performed at a temperature between about 4° C. and about 60° C., between about 10° C. and about 55° C., between about 20° C. and about 50° C., between about 25° C. and about 45° C., between about 30° C. and about 40° C., between about 35° C. and about 39° C., and between about 36° C. and about 38° C. for a predetermined time period of between about 0.5 hours and about 24 hours, between about 1 hour and about 20 hours, between about 1.5 hours and about 16 hours, between about 2 hours and about 12 hours, between about 3 hours and about 10 hours, between about 3.5 hours and about 9 hours, between about 4 hours and about 8 hours, between about 4.5 hours and about 7 hours, between about 5 hours and about 6 hours, and between about 5.25 hours and about 5.75 hours. The incubating may be performed in a humidified environment and air composition resembling an ambient environment. In embodiments, the incubating may be performed at hypoxia (~1-5% O₂), normoxia (~20% O₂), and the like. After incubation, at step 730 of method 700, the removable cap is removed from the second opening of the enrichment bag and a filtration device can be coupled to the spout.

It can be appreciated that steps 735 through 750 of method 700 may be performed similarly to corresponding steps of FIG. 4A. To this end, the enrichment bag may be coupled to a receiving bay waste outlet of a container, the container may be introduced into and coupled to a processing pod, and filtration, isolation, recovery, and detection of pathogen may be subsequently performed. With reference to method 700 of FIG. 7, an embodiment is described wherein any means may be used to apply the pressure differential across the filtration device. For instance, the enrichment bag can be directly connected to a pressure regulator and a vacuum can be pulled to cause the flow of perfusate through the filtration device. In another instance, a positive pressure may be applied to the enrichment bag to compress the enrichment bag and cause perfusate to flow through the filtration device. In yet another instance, the enrichment bag may be placed inside a pressurized chamber, the filtration device may be coupled to an environmental outlet, and a pressure greater than atmospheric pressure can be applied to the pressurized chamber to apply a pressure differential across the filtration device. In this way, growth medium, potentially laden with pathogens, is pushed through the filtration device. As described above, applying the pressure differential may comprise applying a pressure difference across the filtration device of between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4.5 psi and about 5.5 psi. Similar methods of filtration, such as those described in U.S. patent application Ser. No. 17/603,248, which is incorporated herein in its entirety by this reference, may be deployed herein as appropriate.

After filtration, a retentate, which is the portion of the perfusate which did not pass through the filtration device, may then be collected, or recovered, from the filtration device at step 740 of method 700. To collect retentate, the filtration device, or filter(s) thereof, can be introduced into a tube or container with a suitable liquid and sonicated or otherwise have shear applied to it. Subsequently, the retentate is re-suspended in the liquid. Counterflow elution, or backwashing, of a liquid through the filtration device, or filter(s) thereof, may alternatively be deployed. In an embodiment, an appropriate volume of liquid, such as water or a buffered solution, can be pumped backwards, in the direction opposite to the filtration direction used previously, to release the retentate from the filtration device.

In embodiments, about 1-10 ml of deionized water is pumped backwards. In other embodiments, about 1-5 ml of deionized water is pumped backwards. The volume of the liquid can be adjusted based on various factors, for example, the size and/or pore size of the filter(s). In an exemplary embodiment, about 1.5 ml of deionized water is pumped backwards to collect the retentate from the filter(s), which may be about 25 mm in diameter. In other embodiments, the liquid can be, but is not limited to culture medium, a buffered solution, and/or mixtures thereof. Pathogens, which may be microbes, within the retentate can be collected from the liquid.

In embodiments, recovering pathogens from the collected retentate encompasses introducing the collected retentate into a vial or tube suitable for centrifugation. In embodiments, the speed of centrifugation is between about 10 G to about 100 G. In an embodiment, the speed of centrifugation is about 50 G or is about 22 G. In an embodiment, the speed of centrifugation is greater than about 100 G or is less than about 10 G. After centrifugation, the supernatant may be discarded and a pellet from the centrifugation can be processed for detection of pathogens at step 750 of method 700.

Detection at step 750 of method 700 comprises detecting by molecular methods such as selectively culturing the microbe or by polymerase chain reaction (PCR). In embodiments, the detecting can be done by a real-time PCR (RT-PCR), by a quantitative real-time PCR (qRT-PCR), or by a quantitative PCR (qPCR). In embodiments, the processed retentate comprising the pathogen may be assayed for contamination by any other PCR-based detection techniques not listed here. In another embodiment, the processed retentate comprising the pathogen may be assayed by plating on selective media for specific pathogens.

As with method 400, detection during method 700 may provide information to a producer of the food or beverage to quarantine or otherwise remove a product containing the food or beverage from the marketplace. Importantly, the present method permits such a determination to be made in a fraction of the time previously required, enabling producers to readily remove harmful products from the marketplace.

### Enrichment Bag Filling Station

As described above, food or beverage for pathogen detection testing are typically collected in flexible plastic, enrichment bags. Liquid bacterial growth medium is added to the enrichment bags to populate the growth medium with the possible pathogens (e.g., microflora) residing on the food or beverage. To this end, a technician must pour sterile growth medium into the enrichment bags at volumes ranging from 225 milliliters to nearly 4 liters. Especially at higher volumes, it can be challenging to hold the enrichment bag and the liquid-bearing container, or growth medium reservoir, steady so that nothing spills or splashes during the addition process and contaminates the food or beverage. To mitigate this risk, the tip of a funnel or pipettor can be introduced within the enrichment bag to aim the flow of the growth medium. However, even this risks contamination as the funnel is placed into proximity to potentially pathogenic food or beverage samples, which may cross contaminate other samples downstream should the same funnel be used again. Either the funnel must be sterilized between samples, or growth medium must be poured from above the enrichment bags. The time cost of sterilization is prohibitively high in a high-throughput testing setting. Furthermore, having a funnel large enough to accommodate hundreds of milliliters of growth medium usually also means the diameter of the funnel outlet is rather large, which, when filling an enrichment bag full of food, can lead to overflow spillage if the growth medium near the opening of the enrichment bag does not quickly percolate through the food and down into the enrichment bag. Additionally, growth medium can only be poured into a funnel as fast as it exits the funnel, lest the funnel overflow. Dealing with such high volumes of growth medium makes this process slow and attention intensive. Accordingly, the enrichment bag filling station of FIG. 8A through FIG. 12B provides solutions.

As shown in FIG. 8A and FIG. 8B, an enrichment bag filling station 821 comprises an enrichment bag bracket 827, a funnel holder 820, a mounting rack 822, a mounting block 828, and a funnel locking pin 825. As shown in FIG. 8A, a funnel 823, having an inlet at the top and an outlet 824 at the bottom, is mounted to the mounting rack 822 via the mounting block 828 and the funnel holder 820. As shown in FIG. 8B, a plurality of funnels 823 can be mounted to the mounting rack 822 via respective mounting blocks 828 and funnel holders 820. The funnel locking pin 825 can be a permanent locking pin 1125′ or a temporary locking pin 1125″, as shown in FIG. 11A and FIG. 11B, respectively. In each, a funnel 1123 is mounted to a mounting block 1128 via the permanent locking pin 1125′or the temporary locking pin 1125″. Use of the temporary locking pin 1125″ permits easy removal of the funnel 1123 from the enrichment bag filling station to perform e.g., cleaning.

With reference now to FIG. 9A and FIG. 9B, an enrichment bag bracket 927 can be positioned beneath a funnel 923 so as to limit the risk of cross contamination from the funnel 923 entering an enrichment bag 906 mounted in the enrichment bag bracket 927. As discussed earlier, the enrichment bag 906 may be mounted to the enrichment bag bracket 927 via a spout 936 of a second opening of the enrichment bag 906. The spout 936 may comprise a neck configured to rest on the enrichment bag bracket 927. Further, the enrichment bag bracket 927 may have a depression or other feature configured to passively hold the spout 936 of the enrichment bag 906 in a filling position. The filling position may be such that flow from an outlet 924 of the funnel 923 is delivered directly to the enrichment bag 906 via the spout 936 without contact therebetween. To this end, the funnel outlet 924 may be directed toward an opening of the spout 936 of the enrichment bag 906 via a funnel holder 920. The funnel 923 may be hung from funnel locking pin 925, which maybe releasable, allowing the funnel 923 to be removed and cleaned regularly without requiring disassembly. As shown in FIG. 9A and in cartoonized form in FIG. 10, a bottle 926 of growth medium may be placed in the funnel 923 for filling. Owing to the design of the enrichment bag filling station, the bottle 926 of growth medium maybe left unattended in the funnel 923 to drain to completion. In fact, leaving the bottle 926 alone within the funnel 923 is essential to quick operation. If the bottle 926 were to be held over the funnel 923 and poured manually, the funnel 923 would quickly fill entirely.

In other words, the funnel 923 can drain without active management because of the formation of dynamic equilibrium that prevents funnel overflow, as shown in FIG. 10. When the bottle 1026 placed into the funnel 1023, growth medium begins flowing and begins to exit the funnel outlet 1024 and fill the space between the bottle 1026 and the funnel 1023. As the bottle 1026 drains, a vacuum within the bottle 1026 is created, which exerts an upwards force on the remaining growth medium. Air must replace the space left by the evacuated growth medium before more growth medium can flow out. Once the upwards force of the vacuum equilibrates with the downwards force of gravity, growth medium drains preferentially from the space of the funnel 1023 instead of from the bottle 1026. This proceeds until the level of growth medium in the funnel space drops enough that air is permitted to enter the bottle 1026 from the side, resetting the vacuum within the bottle 1026. The space of the funnel 1023 then fills up again, and the process repeats. Because air enters through a different space than the growth medium exits from, laminar flow of the growth medium is maintained and a clean, splash-free pour is executed.

The dynamic equilibrium within the funnel 1023 of FIG. 10 can be controlled, at least in part, by a funnel fitting 1219 as shown in FIG. 12A and FIG. 12B. Controlling the dynamic equilibrium permits flow velocity control according to the risk of overflow. The funnel fitting 1219 shown in FIG. 12A may be placed with a funnel outlet. The funnel fitting 1219 may be sized to maintain a desired flow restriction and flow rate therethrough. To this end, the funnel fitting 1219 have an inner diameter, and outer diameter, and a length from a top of the funnel fitting 1219 to a bottom of the funnel fitting 1219. In embodiments, the inner diameter of the funnel fitting 1219 may be between about 1 mm and bout 10 mm, between about 2 mm and about 9 mm, between about 3 mm and about 8 mm, between about 4 mm and about 7 mm, and/or between about 5 mm and about 6 mm. In embodiments, the length of the funnel fitting 1219 may be between about 5 mm and about 40 mm, between about 10 mm and about 35 mm, between about 15 mm and about and between about 20 mm and about 30 mm. In embodiments, the outer diameter of the funnel fitting may comprise two outer diameters, a first diameter at the top of the funnel fitting 1219 and a second diameter at the bottom of the funnel fitting 1219 to account for the shape of the funnel 1223. To this end, the first diameter may be between about 10 mm and about 40 mm, between about 15 mm and about 35 mm, and between about 20 mm and about 30 mm, and the second diameter may be between about 10 mm and about 40 mm, between about 15 mm and about 35 mm, and between about 20 mm and about 30 mm. FIG. 12B shows exemplary funnel fittings 1219A, 1219B, 1219C, each of which may have different inner diameters and/or lengths.

In embodiments, the funnel fitting 1219 of FIG. 12A and the funnel fittings 1219A, 1219B, 1219C may comprise a material selected from the group consisting of natural rubber, acrylonitrile butadiene styrene, polyethylene, polypropylene, polydimethylsiloxane, and the like.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

### NUMBERED EMBODIMENTS OF THE INVENTION

Notwithstanding the appended claims, the disclosure sets forth the following numbered embodiments:

Embodiment 1. An enrichment bag for food sample processing for pathogen detection, comprising an enclosure configured to contain one or both of a food or beverage sample, the enclosure comprising a first sealable opening at a first end of the enclosure, the first sealable opening defined by opposing side panels of the enclosure and configured to receive the food or beverage sample, and a second sealable opening at a second end of the enclosure and defined at least in part by the opposing side panels of the enclosure, and a spout coupled to the second sealable opening.

Embodiment 1.1. The enrichment bag of Embodiment 1, wherein the second sealable opening comprises an inner dimension that is less than an inner dimension of the first sealable opening.

Embodiment 1.2. The enrichment bag of either Embodiment 1 or 1.1 further comprising a filtration device couplable to the spout, the filtration device being configured to filter one or more particles from the food or beverage sample.

Embodiment 2. The enrichment bag of Embodiment 1.2, wherein the one or more particles comprise a microbe.

Embodiment 2.2. The enrichment bag of any one of Embodiment 1 to 2, wherein the food or beverage sample comprises a leaf vegetable, chicken, beef, pork, swab, cloth, cheese, dairy, spices, or fruit within a growth medium.

Embodiment 3. The enrichment bag of Embodiment 2, wherein the filtration device is further configured to concentrate the microbe from the food or beverage sample.

Embodiment 4. The enrichment bag of any one of Embodiment 1 to 3, wherein the enclosure is configured to be incubated with the food or beverage sample therein.

Embodiment 5. The enrichment bag of Embodiment 1.2, wherein the enclosure is configured to be pressurized with the food or beverage sample and the filtration device therein such that a filtrate from the food or beverage sample flows through the filtration device and out of the enclosure via the second sealable opening.

Embodiment 6. The enrichment bag of any one of Embodiment 1 to 5, wherein the enclosure comprises a material selected from the group consisting of polypropylene, polyethylene, and low-density polyethylene.

Embodiment 7. The enrichment bag of any one of Embodiment 1 to 6, wherein the enclosure comprises a rectangular shape.

Embodiment 8. The enrichment bag of any one of Embodiment 1 to 7, wherein the enclosure is configured to receive a food or beverage sample comprising up to about 25 grams, up to 60 grams, up to about 350 grams, up to 375 grams, or up to about 1500 grams of a leaf vegetable, chicken, beef, pork, swab, cloth, cheese, dairy, spices, or fruit.

Embodiment 9. The enrichment bag of any one of Embodiment 1 to 8, wherein the first sealable opening is configured to be permanently sealed with heat.

Embodiment 10. The enrichment bag of any one of Embodiment 1 to 9, wherein the first sealable opening is configured to be temporarily or permanently sealed with a tin tie or a zipper channel.

Embodiment 11. The enrichment bag of Embodiment 1.1, wherein the inner dimension of the first sealable opening and the inner dimension of the second sealable opening each comprise an inner width or inner diameter.

Embodiment 12. The enrichment bag of Embodiment 11, wherein the inner dimension of the first sealable opening is an inner width having a distance of between about 1 cm and about 10 cm.

Embodiment 13. The enrichment bag of Embodiment 11 or 12, wherein the inner dimension of the first sealable opening is an inner width having a distance of between about 5% and about 95% of an outer width of the first sealable opening.

Embodiment 14. The enrichment bag of any one of Embodiment 11 to 13, wherein the inner dimension of the second sealable opening is an inner width having a distance of between about 1 mm and about 10 cm.

Embodiment 14.1. The enrichment bag of any one of Embodiment 11 to 14, wherein the inner dimension of the second sealable opening is an inner width having a distance of between about 5% and about 95% of an outer width of the second sealable opening.

Embodiment 14.2. The enrichment bag of any one of Embodiment 11 to 14, wherein the inner dimension of the second sealable opening is an inner diameter and the inner diameter is between about 1 mm and about 1 cm.

Embodiment 15. The enrichment bag of any one of Embodiment 1 to 14.2, wherein the second sealable opening is on a corner of the second end of the enclosure.

Embodiment 16. The enrichment bag of any one of Embodiment 1 to 15, wherein the second sealable opening is sealable about a perimeter of the spout.

Embodiment 17. The enrichment bag of any one of Embodiment 1 to 16, wherein the spout comprises a removable cap.

Embodiment 18. The enrichment bag of any one of Embodiment 1 to 17, wherein the spout comprises a first spout and the removable cap comprises a first removable cap, the enclosure further comprising a third sealable opening comprising an inner dimension that is less than an inner dimension of the first sealable opening, and a second spout coupled to the third sealable opening and comprising a second removable cap.

Embodiment 19. The enrichment bag of Embodiment 18, wherein the third sealable opening is configured to be sealed about a perimeter of the second spout.

Embodiment 20. The enrichment bag of any one of Embodiment 1 to 19, wherein an inner dimension of second opening is about equal to an outer dimension of the spout.

Embodiment 21. The enrichment bag of Embodiment 20, wherein the outer dimension comprises an outer width or outer diameter of the spout.

Embodiment 22. The enrichment bag of Embodiment 17, wherein the second sealable opening is configured to be temporarily sealed via the removable cap.

Embodiment 23. The enrichment bag of Embodiment 17, wherein the spout and the removable cap are configured to be releasably coupled by a rotational coupling or a press fit coupling.

Embodiment 24. The enrichment bag of any one of Embodiment 1 to 23, wherein the spout has a shape selected from the group consisting of circular, rectangular, and elliptical.

Embodiment 25. The enrichment bag of any one of Embodiment 1 to 24, wherein the spout comprises a material selected from the group consisting of polypropylene, polyethylene, and low density polyethylene.

Embodiment 26. The enrichment bag of any one of Embodiment 1 to 25, wherein the spout comprises a spout neck.

Embodiment 26.1. The enrichment bag of Embodiment 26, wherein the enclosure is configured to releasably hang from the spout neck.

Embodiment 27. A method for food sample processing for pathogen detection, comprising introducing a food or beverage into an enclosure configured to contain one or both of a food or beverage sample, the food or beverage sample comprising the food or beverage and a growth medium, the enclosure comprising a first sealable opening at a first end of the enclosure, the first sealable opening defined by opposing side panels of the enclosure and configured to receive the food or beverage, and a second sealable opening at a second end of the enclosure and defined at least in part by the opposing side panels of the enclosure, introducing the growth medium into the enclosure via a spout coupled to the second sealable opening, incubating the enclosure containing the food or beverage sample and the growth medium, and extracting a fluid sample from the enclosure via the spout.

Embodiment 27.1. The method of Embodiment 27 further comprising detecting a presence of pathogen within the extracted fluid sample.

Embodiment 27.2. A method for food sample processing for pathogen detection, comprising introducing a food or beverage into an enclosure configured to contain one or both of a food or beverage sample, the food or beverage sample comprising the food or beverage and a growth medium, the enclosure comprising a first sealable opening at a first end of the enclosure, the first sealable opening defined by opposing side panels of the enclosure and configured to receive the food or beverage, and a second sealable opening at a second end of the enclosure and defined at least in part by the opposing side panels of the enclosure, introducing the growth medium into the enclosure via a spout coupled to the second sealable opening, incubating the enclosure containing the food or beverage sample, coupling a filtration device to the spout of enclosure, applying a pressure differential across the filtration device, and extracting a retentate from the filtration device.

Embodiment 27.3. The method of Embodiment 27.2 further comprising detecting a pathogen within the retentate extracted from the filtration device.

Embodiment 28. The method of Embodiment 27 or 27.2, wherein introducing the growth medium into the enclosure comprises hanging the enclosure from an enclosure stand by a neck of the spout.

Embodiment 29. The method of Embodiment 27 further comprising introducing a filtration device into the enclosure via the spout, wherein the filtration device is configured to filter one or more particles from the food or beverage sample.

Embodiment 30. The method of Embodiment 29 further comprising filtering the one or more particles from food or beverage sample via the filtration device.

Embodiment 31. The method of Embodiment 27 further comprising coupling a filtration device to the spout, wherein the filtration device is configured to filter one or more particles from the food or beverage sample.

Embodiment 31.1. The method of Embodiment 30 or 31, wherein the filtering comprises pressurizing the enclosure with the food or beverage sample and the filtration device therein such that a portion of the food or beverage sample flows through the filtration device and out of the enclosure via the second sealable opening.

Embodiment 32. The method of Embodiment 31 further comprising filtering the one or more particles from the food or beverage sample via the filtration device.

Embodiment 32.1. The method of Embodiment 32, wherein the one or more particles comprise a microbe.

Embodiment 33. The method of Embodiment 32 further comprising concentrating the microbe with the filtration device.

Embodiment 34. The method of Embodiment 27, wherein the spout comprises a first spout, the enclosure further comprising a third sealable opening of the enclosure body comprising an inner dimension that is less than the inner dimension of the first sealable opening, the third sealable opening comprising a second spout coupled thereto, and wherein the growth medium is introduced into the enclosure via the first and second spouts simultaneously.

Embodiment 35. The method of Embodiment 27 or 27.2 further comprising, prior to introducing the growth medium, permanently sealing first sealable opening with heat.

Embodiment 36. The method of Embodiment 27 or 27.2 further comprising, prior to introducing the growth medium, temporarily or permanently sealing the second sealable opening with a tin tie or a zipper channel.

Embodiment 37. The method of Embodiment 27 or 27.2 further comprising, prior to the incubating, temporarily sealing the second sealable opening by releasably coupling a removable spout cap to the spout.

Embodiment 38. The method of Embodiment 27 or 27.2, wherein an inner dimension of the first sealable opening and an inner dimension of a second sealable opening each comprise an inner width or inner diameter.

Embodiment 39. The method of Embodiment 27 or 27.2, wherein the method is automated.

Embodiment 40. The method of Embodiment 27 or 27.2 further comprising sealing the second sealable opening around the spout with heat.

Embodiment 41. The method of Embodiment 27 or 27.2 further comprising reversibly sealing the second sealable opening with a tin tie or a zipper channel.

Embodiment 42. The method of Embodiment 27 or 27.2 further comprising reversibly sealing the second sealable opening by releasably coupling a removable spout cap to the spout.

Embodiment 43. The method of Embodiment 27 or 27.2, wherein the incubating is performed at a temperature between about 4° C. and about 60° C., between about 10° C. and about 55° C., between about 20° C. and about 50° C., between about 25° C. and about 45° C., between about 30° C. and about 40° C., between about 35° C. and about 39° C., and between about 36° C. and about 38° C.

Embodiment 43.1. The method of Embodiment 27 or 27.2, wherein the incubating is performed for between about 0.5 hours and about 24 hours, between about 1 hour and about 20 hours, between about 1.5 hours and about 16 hours, between about 2 hours and about 12 hours, between about 3 hours and about 10 hours, between about 3.5 hours and about 9 hours, between about 4 hours and about 8 hours, between about 4.5 hours and about 7 hours, between about 5 hours and about 6 hours, and between about 5.25 hours and about 5.75 hours.

Embodiment 43.2. The method of Embodiment 27 or 27.2, wherein applying a pressure differential comprises applying a pressure difference across the filtration device of between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4. 5 psi and about 5.5 psi.

Embodiment 43.3. The method of Embodiment 27 or 27.2, wherein extracting the retentate comprises backwashing the filtration device with a predetermined volume of washing fluid.

Embodiment 43.4. The method of Embodiment 27.1 or 27.3, wherein detecting the pathogen comprises performing one or more of polymerase chain reaction (PCR), real-time PCR, and quantitative real-time PCR.

Embodiment 44. The method of Embodiment 27 or 27.2, wherein the food or beverage comprises leaf vegetable, chicken, beef, pork, swab, cloth, cheese, dairy, spices, or fruit within the growth medium.

Embodiment 45. A container for enrichment bags for food sample processing for pathogen detection, comprising a container base including two or more enrichment bag receiving bays, each receiving bay being separated by a barrier, each receiving bay comprising a waste conduit outlet, and a waste conduit in fluid communication with each waste conduit outlet and with an external environment.

Embodiment 45.1. The container of Embodiment 45, wherein the barrier prevents contamination between receiving bays.

Embodiment 45.2. The container of Embodiment 45, wherein the two or more enrichment bag receiving bays comprises two, three, four, five, or six receiving bays.

Embodiment 46. The container of any one of Embodiment 45 to 45.2 further comprising a lid.

Embodiment 47. The container of any one of Embodiment 45 to 46, wherein a dimension of the waste conduit is pre-determined based on a food or beverage capacity of each of one or more enrichment bags within the receiving bays in order to control a flow rate of perfusate through a fluid filtration device coupled to each of the one or more enrichment bags.

Embodiment 48. The container of Embodiment 47, wherein the dimension is at least one of a length and a diameter.

Embodiment 49. The container of any one of Embodiment 45 to 48, wherein the waste conduit may be made from a material selected from the group consisting of polypropylene, polytetrafluoroethylene, expanded polytetrafluoroethylene, polyethylene, and low-density polyethylene.

Embodiment 50. A system for food sample processing for pathogen detection, comprising an array of processing pods, each of the processing pods comprising a tubular cavity configured to receive a container, each of the processing pods comprising a fluid inlet and a fluid outlet, each of the processing pods being sealable and configured to have a pressure applied therein, and a plurality of containers, each container comprising a container base including two or more enrichment bag receiving bays, each receiving bay comprising a waste conduit outlet; and a waste conduit in fluid communication with each waste conduit outlet and with an external environment via the fluid outlet of each processing pod.

Embodiment 51. The system of Embodiment 50, wherein each processing pod comprises a sealable door at an end of the tubular cavity.

Embodiment 52. The system of Embodiment 50 or 51, wherein each receiving bay of each container is separated by a barrier.

Embodiment 53. The system of Embodiment 52, wherein the barrier prevents contamination between receiving bays.

Embodiment 54. The system of any one of Embodiment 50 to 53, wherein the two or more enrichment bag receiving bays comprises two, three, four, five, or six receiving bays.

Embodiment 55. The system of any one of Embodiment 50 to 54, wherein each container further comprises a lid.

Embodiment 56. The system of any one of Embodiment 50 to 55, wherein a dimension of the waste conduit of each container is pre-determined based on a food or beverage capacity of each of one or more enrichment bags within the receiving bays in order to control a flow rate of perfusate through a fluid filtration device coupled to each of the one or more enrichment bags.

Embodiment 57. The system of Embodiment 56, wherein the dimension is at least one of a length and a diameter.

Embodiment 58. The system of any one of Embodiment 50 to 57, wherein the waste conduit of each container may be made from a material selected from the group consisting of polyvinylchloride, acrylonitrile butadiene styrene, polyvinylidene difluoride, polyethylene terephthalate, polypropylene, polytetrafluoroethylene, expanded polytetrafluoroethylene, low-density polyethylene, and high-density polyethylene.

Embodiment 59. The system of any one of Embodiment 50 to 58 further comprising two or more enrichment bags within a respective receiving bay of each container.

Embodiment 60. The system of Embodiment 59, wherein each of the two or more enrichment bags is fluidically connected to the waste conduit outlet of the respective receiving bay.

Embodiment 61. A method for using a system for food sample processing for pathogen detection, comprising introducing a food or beverage into an enrichment bag including an enclosure comprising a first sealable opening at a first end of the enclosure and a second sealable opening at a second end of the enclosure, wherein the first sealable opening is defined by opposing side panels of the enclosure and is configured to receive a food or beverage sample comprising the food or beverage and a growth medium, and the second sealable opening is defined at least in part by the opposing side panels of the enclosure, introducing the growth medium into the enclosure via a spout coupled to the second sealable opening, incubating the enclosure containing the food or beverage sample and the growth medium, coupling a filtration device to the spout, positioning the enrichment bag within an enrichment bag receiving bay of a container, fitting the container within a processing pod of an array of processing pods, applying a pressure differential across the filtration device, and extracting a retentate from the filtration device.

Embodiment 62. The method of Embodiment 61, wherein the incubating is performed when the container is fitted within the processing pod.

Embodiment 63. The method of Embodiment 61 or 62, wherein the processing pod comprises a tubular cavity.

Embodiment 64. The method of any one of Embodiment 61 to 63, wherein positioning the enrichment bag within the enrichment bag receiving bay comprises coupling the enrichment bag with a container waste conduit via a waste conduit outlet.

Embodiment 65. The method of any one of Embodiment 61 to 64, wherein the fitting comprises sealing a sealable door at an end of the processing pod.

Embodiment 66. The method of any one of Embodiment 61 to 65, wherein the fitting comprises fluidically coupling the container waste conduit with an external environment via a processing pod outlet waste outlet.

Embodiment 67. The method of any one of Embodiment 61 to 66, wherein the incubating is performed at a temperature between about 4° C. and about 60° C., between about 10° C. and about 55° C., between about 20° C. and about 50° C., between about 25° C. and about 45° C., between about 30° C. and about 40° C., between about 35° C. and about 39° C., and between about 36° C. and about 38° C.

Embodiment 67.1. The method of any one of Embodiment 61 to 67, wherein the incubating is performed for between 0.5 hours and about 24 hours, between about 1 hour and about 20 hours, between about 1.5 hours and about 16 hours, between about 2 hours and about 12 hours, between about 3 hours and about 10 hours, between about 3.5 hours and about 9 hours, between about 4 hours and about 8 hours, between about 4.5 hours and about 7 hours, between about 5 hours and about 6 hours, and between about 5.25 hours and about 5.75 hours.

Embodiment 68. The method of any one of Embodiment 61 to 67.1, wherein the food or beverage sample comprises a leaf vegetable, chicken, beef, pork, swab, cloth, cheese, dairy, spices, or fruit within the growth medium.

Embodiment 69. The method of any one of Embodiment 61 to 68, wherein applying a pressure differential comprises applying a pressure difference across the filtration device of between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4.5 psi and about 5.5 psi.

Embodiment 70. The method of any one of Embodiment 61 to 69, wherein extracting the retentate comprises backwashing the filtration device with a predetermined volume of washing fluid.

Embodiment 71. The method of any one of Embodiment 61 to 70 further comprising a plurality of enrichment bags within respective receiving bays of each container.

Embodiment 72. The method of any one of Embodiment 61 to 71, further comprising detecting a pathogen within the retentate extracted from the filtration device.

Embodiment 73. The method of Embodiment 72, wherein the detecting comprises performing one or more of polymerase chain reaction (PCR), real-time PCR, and quantitative real-time PCR.

We claim:

1. An enrichment bag for food sample processing for pathogen detection, comprising:
an enclosure configured to contain one or both of a food or beverage sample, the enclosure comprising
a first sealable opening at a first end of the enclosure, the first sealable opening defined by opposing side panels of the enclosure and configured to receive the food or beverage sample, and
a second sealable opening at a second end of the enclosure and defined at least in part by the opposing side panels of the enclosure; and
a spout coupled to the second sealable opening.

2. The enrichment bag of claim 1, wherein the second sealable opening comprises an inner dimension that is less than an inner dimension of the first sealable opening.

3. The enrichment bag of claim 1 further comprising a filtration device couplable to the spout, the filtration device being configured to filter one or more particles from the food or beverage sample.

4. The enrichment bag of claim 3, wherein the one or more particles comprise a microbe.

5. The enrichment bag of claim 1, wherein the food or beverage sample comprises a leaf vegetable, chicken, beef, pork, swab, cloth, cheese, dairy, spices, or fruit within a growth medium.

6. The enrichment bag of claim 4, wherein the filtration device is further configured to concentrate the microbe from the food or beverage sample.

7. The enrichment bag of claim 1, wherein the enclosure is configured to be incubated with the food or beverage sample therein.

8. The enrichment bag of claim 3, wherein the enclosure is configured to be pressurized with the food or beverage sample and the filtration device therein such that a filtrate from the food or beverage sample flows through the filtration device and out of the enclosure via the second sealable opening.

9. The enrichment bag of claim 1, wherein the enclosure comprises a material selected from the group consisting of polypropylene, polyethylene, and low-density polyethylene.

10. The enrichment bag of claim 1, wherein the enclosure comprises a rectangular shape.

11. The enrichment bag of claim 1, wherein the enclosure is configured to receive a food or beverage sample comprising up to about 25 grams, up to 60 grams, up to about 350 grams, up to 375 grams, or up to about 1500 grams of leaf vegetable, chicken, beef, pork, swab, cloth, cheese, dairy, spices, or fruit.

12. The enrichment bag of claim 1, wherein the first sealable opening is configured to be permanently sealed with heat.

13. The enrichment bag of claim 1, wherein the first sealable opening is configured to be temporarily or permanently sealed with a tin tie or a zipper channel.

14. The enrichment bag of claim 2, wherein the inner dimension of the first sealable opening and the inner dimension of the second sealable opening each comprise an inner width or inner diameter.

15. The enrichment bag of claim 14, wherein the inner dimension of the first sealable opening is an inner width having a distance of between about 1 cm and about 10 cm.

16. The enrichment bag of claim 14, wherein the inner dimension of the first sealable opening is an inner width having a distance of between about 5% and about 95% of an outer width of the first sealable opening.

17. The enrichment bag of claim 14, wherein the inner dimension of the second sealable opening is an inner width having a distance of between about 1 mm and about 10 cm.

18. The enrichment bag of claim 14, wherein the inner dimension of the second sealable opening is an inner width having a distance of between about 5% and about 95% of an outer width of the second sealable opening.

19. The enrichment bag of claim 14, wherein the inner dimension of the second sealable opening is an inner diameter and the inner diameter is between about 1 mm and about 1 cm.

20. The enrichment bag of claim 1, wherein the second sealable opening is on a corner of the second end of the enclosure.

21. The enrichment bag of claim 1, wherein the second sealable opening is sealable about a perimeter of the spout.

22. The enrichment bag of claim 1, wherein the spout comprises a removable cap.

23. The enrichment bag of claim 1, wherein the spout comprises a first spout and the removable cap comprises a first removable cap, the enclosure further comprising:
a third sealable opening comprising an inner dimension that is less than an inner dimension of the first sealable opening; and
a second spout coupled to the third sealable opening and comprising a second removable cap.

24. The enrichment bag of claim 23, wherein the third sealable opening is configured to be sealed about a perimeter of the second spout.

25. The enrichment bag of claim 1, wherein an inner dimension of second opening is about equal to an outer dimension of the spout.

26. The enrichment bag of claim 25, wherein the outer dimension comprises an outer width or outer diameter of the spout.

27. The enrichment bag of claim 22, wherein the second sealable opening is configured to be temporarily sealed via the removable cap.

28. The enrichment bag of claim 22, wherein the spout and the removable cap are configured to be releasably coupled by a rotational coupling or a press fit coupling.

29. The enrichment bag of claim 1, wherein the spout has a shape selected from the group consisting of circular, rectangular, and elliptical.

30. The enrichment bag of claim 1, wherein the spout comprises a material selected from the group consisting of polypropylene, polyethylene, and low density polyethylene.

31. The enrichment bag of claim 1, wherein the spout comprises a spout neck.

32. The enrichment bag of claim 26, wherein the enclosure is configured to releasably hang from the spout neck.

33. A method for food sample processing for pathogen detection, comprising:
introducing a food or beverage into an enclosure configured to contain one or both of a food or beverage sample, the food or beverage sample comprising the food or beverage and a growth medium, the enclosure comprising a first sealable opening at a first end of the enclosure, the first sealable opening defined by opposing side panels of the enclosure and configured to receive the food or beverage, and a second sealable opening at a second end of the enclosure and defined at least in part by the opposing side panels of the enclosure;
introducing the growth medium into the enclosure via a spout coupled to the second sealable opening;
incubating the enclosure containing the food or beverage sample and the growth medium; and
extracting a fluid sample from the enclosure via the spout.

34. The method of claim 33 further comprising detecting a presence of pathogen within the extracted fluid sample.

35. The method of claim 33, wherein introducing the growth medium into the enclosure comprises
hanging the enclosure from an enclosure stand by a neck of the spout.

36. The method of claim 33 further comprising introducing a filtration device into the enclosure via the spout, wherein the filtration device is configured to filter one or more particles from the food or beverage sample.

37. The method of claim 36 further comprising filtering the one or more particles from the food or beverage sample via the filtration device.

38. The method of claim 33 further comprising
coupling a filtration device to the spout, wherein the filtration device is configured to filter one or more particles from the food or beverage sample.

39. The method of claim 36, wherein the filtering comprises pressurizing the enclosure with the food or beverage sample and the filtration device therein such that a portion of the food or beverage sample flows through the filtration device and out of the enclosure via the second sealable opening.

40. The method of claim 39 further comprising filtering the one or more particles from the food or beverage sample via the filtration device.

41. The method of claim 40, wherein the one or more particles comprise a microbe.

42. The method of claim 40 further comprising concentrating the microbe with the filtration device.

43. The method of claim 33, wherein the spout comprises a first spout, the enclosure further comprising a third sealable opening of the enclosure body comprising an inner dimension that is less than the inner dimension of the first sealable opening, the third sealable opening comprising a second spout coupled thereto, and
wherein the growth medium is introduced into the enclosure via the first and second spouts simultaneously.

44. The method of claim 33 further comprising, prior to introducing the growth medium, permanently sealing first sealable opening with heat.

45. The method of claim 33 further comprising, prior to introducing the growth medium, temporarily or permanently sealing the second sealable opening with a tin tie or a zipper channel.

46. The method of claim 33 further comprising, prior to the incubating, temporarily sealing the second sealable opening by releasably coupling a removable spout cap to the spout.

47. The method of claim 33, wherein an inner dimension of the first sealable opening and an inner dimension of a second sealable opening each comprise an inner width or inner diameter.

48. The method of claim 33, wherein the method is automated.

49. The method of claim 33 further comprising sealing the second sealable opening around the spout with heat.

50. The method of claim 33 further comprising reversibly sealing the second sealable opening with a tin tie or a zipper channel.

51. The method of claim 33 further comprising reversibly sealing the second sealable opening by releasably coupling a removable spout cap to the spout.

52. The method of claim 33, wherein the incubating is performed at a temperature between about 4° C. and about 60° C., between about 10° C. and about 55° C., between about 20° C. and about 50° C., between about 25° C. and about 45° C., between about 30° C. and about 40° C., between about 35° C. and about 39° C., and between about 36° C. and about 38° C.

53. The method of claim 33, wherein the incubating is performed for between about 0.5 hours and about 24 hours, between about 1 hour and about 20 hours, between about 1.5 hours and about 16 hours, between about 2 hours and about 12 hours, between about 3 hours and about 10 hours, between about 3.5 hours and about 9 hours, between about 4 hours and about 8 hours, between about 4.5 hours and about 7 hours, between about 5 hours and about 6 hours, and between about 5.25 hours and about 5.75 hours.

54. The method of claim 33, wherein applying a pressure differential comprises applying a pressure difference across the filtration device of between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4.5 psi and about 5.5 psi.

55. The method of claim 33, wherein extracting the retentate comprises backwashing the filtration device with a predetermined volume of washing fluid.

56. The method of claim 34, wherein detecting the pathogen comprises performing one or more of polymerase chain reaction (PCR), real-time PCR, and quantitative real-time PCR.

57. The method of claim 33, wherein the food or beverage sample comprises a leaf vegetable, chicken, beef, pork, swab, cloth, cheese, dairy, spices, or fruit within the growth medium.

58. A container for enrichment bags for food sample processing for pathogen detection, comprising:
a container base including two or more enrichment bag receiving bays, each receiving bay being separated by a barrier, each receiving bay comprising a waste conduit outlet; and
a waste conduit in fluid communication with each waste conduit outlet and with an external environment.

59. The container of claim 58, wherein the barrier prevents contamination between receiving bays.

60. The container of claim 58, wherein the two or more enrichment bag receiving bays comprises two, three, four, five, or six receiving bays.

61. The container of claim 58 further comprising a lid.

62. The container of claim 58, wherein a dimension of the waste conduit is pre-determined based on a food or beverage capacity of each of one or more enrichment bags within the receiving bays in order to control a flow rate of perfusate through a fluid filtration device coupled to each of the one or more enrichment bags.

63. The container of claim 62, wherein the dimension is at least one of a length and a diameter.

64. The container of claim 58, wherein the waste conduit may be made from a material selected from the group consisting of polypropylene, polytetrafluoroethylene, expanded polytetrafluoroethylene, polyethylene, and low-density polyethylene.

65. A method for food sample processing for pathogen detection, comprising:
introducing a food or beverage into an enclosure configured to contain one or both of a food or beverage sample, the food or beverage sample comprising the food or beverage and a growth medium, the enclosure comprising a first sealable opening at a first end of the enclosure, the first sealable opening defined by opposing side panels of the enclosure and configured to receive the food or beverage, and a second sealable opening at a second end of the enclosure and defined at least in part by the opposing side panels of the enclosure;
introducing the growth medium into the enclosure via a spout coupled to the second sealable opening;
incubating the enclosure containing the food or beverage sample;
coupling a filtration device to the spout of enclosure;
applying a pressure differential across the filtration device; and
extracting a retentate from the filtration device.

66. The method of claim 65 further comprising detecting a pathogen within the retentate extracted from the filtration device.

67. The method of claim 65, wherein introducing the growth medium into the enclosure comprises hanging the enclosure from an enclosure stand by a neck of the spout.

68. The method of claim 65 further comprising, prior to introducing the growth medium, permanently sealing first sealable opening with heat.

69. The method of claim 65 further comprising, prior to introducing the growth medium, temporarily or permanently sealing the second sealable opening with a tin tie or a zipper channel.

70. The method of claim 65 further comprising, prior to the incubating, temporarily sealing the second sealable opening by releasably coupling a removable spout cap to the spout.

71. The method of claim 65, wherein an inner dimension of the first sealable opening and an inner dimension of a second sealable opening each comprise an inner width or inner diameter.

72. The method of claim 65, wherein the method is automated.

73. The method of claim 65 further comprising sealing the second sealable opening around the spout with heat.

74. The method of claim 65 further comprising reversibly sealing the second sealable opening with a tin tie or a zipper channel.

75. The method of claim 65 further comprising reversibly sealing the second sealable opening by releasably coupling a removable spout cap to the spout.

76. The method of claim 65, wherein the incubating is performed at a temperature between about 4° C. and about 60° C., between about 10° C. and about 55° C., between about 20° C. and about 50° C., between about 25° C. and about 45° C., between about 30° C. and about 40° C., between about 35° C. and about 39° C., and between about 36° C. and about 38°C.

77. The method of claim 65, wherein the incubating is performed for between about 0.5 hours and about 24 hours, between about 1 hour and about 20 hours, between about 1.5 hours and about 16 hours, between about 2 hours and about 12 hours, between about 3 hours and about 10 hours, between about 3.5 hours and about 9 hours, between about 4 hours and about 8 hours, between about 4.5 hours and about 7 hours, between about 5 hours and about 6 hours, and between about 5.25 hours and about 5.75 hours.

78. The method of claim 65, wherein applying a pressure differential comprises applying a pressure difference across the filtration device of between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4.5 psi and about 5.5 psi.

79. The method of claim 65, wherein extracting the retentate comprises backwashing the filtration device with a predetermined volume of washing fluid.

80. The method of claim 66, wherein detecting the pathogen comprises performing one or more of polymerase chain reaction (PCR), real-time PCR, and quantitative real-time PCR.

81. The method of claim 65, wherein the food or beverage sample comprises a leaf vegetable, chicken, beef, pork, swab, cloth, cheese, dairy, spices, or fruit within the growth medium.

82. A system for food sample processing for pathogen detection, comprising:
an array of processing pods, each of the processing pods comprising a tubular cavity configured to receive a container, each of the processing pods comprising a fluid inlet and a fluid outlet, each of the processing pods being sealable and configured to have a pressure applied therein; and
a plurality of containers, each container comprising
a container base including two or more enrichment bag receiving bays, each receiving bay comprising a waste conduit outlet; and
a waste conduit in fluid communication with each waste conduit outlet and with an external environment via the fluid outlet of each processing pod.

83. The system of claim 82, wherein each processing pod comprises a sealable door at an end of the tubular cavity.

84. The system of claim 82, wherein each receiving bay of each container is separated by a barrier.

85. The system of claim 84, wherein the barrier prevents contamination between receiving bays.

86. The system of claim 82, wherein the two or more enrichment bag receiving bays comprises two, three, four, five, or six receiving bays.

87. The system of claim 82, wherein each container further comprises a lid.

88. The system of claim 82, wherein a dimension of the waste conduit of each container is pre-determined based on a food or beverage capacity of each of one or more enrichment bags within the receiving bays in order to control a flow rate of perfusate through a fluid filtration device coupled to each of the one or more enrichment bags.

89. The system of claim 88, wherein the dimension is at least one of a length and a diameter.

90. The system of claim 82, wherein the waste conduit of each container may be made from a material selected from the group consisting of polyvinylchloride, acrylonitrile butadiene styrene, polyvinylidene difluoride, polyethylene terephthalate, polypropylene, polytetrafluoroethylene, expanded polytetrafluoroethylene, low-density polyethylene, and high-density polyethylene.

91. The system of claim 82 further comprising two or more enrichment bags within a respective receiving bay of each container.

92. The system of claim 91, wherein each of the two or more enrichment bags is fluidically connected to the waste conduit outlet of the respective receiving bay.

93. A method for using a system for food sample processing for pathogen detection, comprising:
introducing a food or beverage into an enrichment bag including an enclosure comprising a first sealable opening at a first end of the enclosure and a second sealable opening at a second end of the enclosure, wherein the first sealable opening is defined by opposing side panels of the enclosure and is configured to receive a food or beverage sample comprising the food or beverage and a growth medium, and the second sealable opening is defined at least in part by the opposing side panels of the enclosure;
introducing the growth medium into the enclosure via a spout coupled to the second sealable opening;
incubating the enclosure containing the food or beverage sample and the growth medium;
coupling a filtration device to the spout;
positioning the enrichment bag within an enrichment bag receiving bay of a container;
fitting the container within a processing pod of an array of processing pods;
applying a pressure differential across the filtration device; and
extracting a retentate from the filtration device.

94. The method of claim 93, wherein the incubating is performed when the container is fitted within the processing pod.

95. The method of claim 93, wherein the processing pod comprises a tubular cavity.

96. The method of claim 93, wherein positioning the enrichment bag within the enrichment bag receiving bay comprises coupling the enrichment bag with a container waste conduit via a waste conduit outlet.

97. The method of claim 93, wherein the fitting comprises sealing a sealable door at an end of the processing pod.

98. The method of claim 93, wherein the fitting comprises fluidically coupling the container waste conduit with an external environment via a processing pod outlet waste outlet.

99. The method of claim 93, wherein the incubating is performed at a temperature between about 4° C. and about 60° C., between about 10° C. and about 55° C., between about 20° C. and about 50° C., between about 25° C. and about 45° C., between about 30° C. and about 40°C., between about 35° C. and about 39° C., and between about 36° C. and about 38° C.

100. The method of claim 93, wherein the incubating is performed for between 0.5 hours and about 24 hours, between about 1 hour and about 20 hours, between about 1.5 hours and about 16 hours, between about 2 hours and about 12 hours, between about 3 hours and about 10 hours, between about 3.5 hours and about 9 hours, between about 4 hours and about 8 hours, between about 4.5 hours and about 7 hours, between about 5 hours and about 6 hours, and between about 5.25 hours and about 5.75 hours.

101. The method of claim 93, wherein the food or beverage sample comprises a leaf vegetable, chicken, beef, pork, swab, cloth, cheese, dairy, spices, or fruit within the growth medium.

102. The method of claim 93, wherein applying a pressure differential comprises applying a pressure difference across the filtration device of between about 1 psi and 50 psi, between about 1.5 psi and about 40 psi, between about 2 psi and about 30 psi, between about 2.5 psi and about 20 psi, between about 3 psi and about 20 psi, between about 3.5 psi and about 10 psi, between about 4 psi and about 7.5 psi, between about 4.5 psi and about 5.5 psi.

103. The method of claim 93, wherein extracting the retentate comprises backwashing the filtration device with a predetermined volume of washing fluid.

104. The method of claim 93 further comprising a plurality of enrichment bags within respective receiving bays of each container.

105. The method of claim 93, further comprising detecting a pathogen within the retentate extracted from the filtration device.

106. The method of claim 105, wherein the detecting comprises performing one or more of polymerase chain reaction (PCR), real-time PCR, and quantitative real-time PCR.
